(19) United States
(12) Patent Application Publication
(10) Pub. No.: US 2017/0095440 A1
(43) Pub. Date: Apr. 6, 2017

(54) METHOD OF TREATING CANINE PANCREATITIS

(71) Applicant: CK9, LLC, Kansas City, KS (US)

(72) Inventors: Long-Shiuh CHEN, Lake Forest, CA (US); Diana WOOD, Shawnee, KS (US); Aidan NUTTALL, Carlsbad, CA (US)

(21) Appl. No.: 15/314,697

(22) PCT Filed: May 29, 2015
(86) PCT No.: PCT/US2015/033237

§ 371 Date: Nov. 29, 2016

Related U.S. Application Data
(60) Provisional application No. 62/005,070, filed on May 30, 2014.

(51) Int. Cl. A61K 31/245; A61K 9/48; A61K 9/00

(52) U.S. Cl. CPC A61K 31/245; A61K 9/0053; A61K 9/48

(57) ABSTRACT

A method for treating pancreatitis that involves identifying a patient with pancreatitis and administering to the patient a daily dose of Camostat Mesilate of between about 8 to about 1300 mg/kg or an equivalent amount of or 4-(4-guan-idino-benzoyloxy) phenylacetic acid is disclosed. The dosage can be administered as a single dose or can be administered in 2, 3, 4 or more doses to achieve the daily dose. The treatment can be given for a week (7 days), a month (31 days) or several months, or for as long as the treatment provides an increased quality of life. The drug can be administered orally in an oral capsule. The methods can be conveniently used in the treatment of canine pancreatitis by measuring the serum concentration of canine pancreas-specific lipase and administering a dose of the drug when the dog has a serum concentration of about 400 μg/L or more of canine pancreas-specific lipase.

### BACKGROUND OF THE INVENTION

Chronic pancreatitis is a chronic inflammatory condition of the pancreas that is characterized by irreversible damage to the parenchyma in conjunction with loss of exocrine, and ultimately endocrine, function accompanied by histologic evidence of fibrosis and atrophy. Clinically, chronic pancreatitis is associated with lethargy, a decreased appetite, and/or vomiting but it can often remain subclinical, leading to infrequent diagnosis and an underestimation of its prevalence in the clinical context. Due to a complete lack of approved pharmaceutical agents, treatment of canine chronic pancreatitis is limited to supportive and even palliative care. Recent publications based on histopathology evaluations have reported evidence for chronic pancreatitis in 34% to 64% of unselected patients submitted for necropsy. The etiology and pathogenesis of canine chronic pancreatitis remains incompletely understood, but possible risk factors include genetic and hereditary predisposition and hypertriglyceridemia. In many cases, however, no risk factors can be identified and the etiology remains unclear.

Camostat Mesilate (N,N-dimethylcarbamoylmethyl-4-(4-guanidinobenzoyloxy)phenylacetate methanesulfonate (Formula I) is a synthetic serine

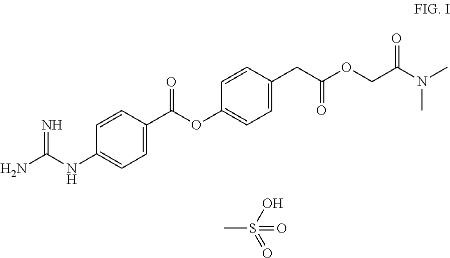

protease inhibitor with pronounced anti-trypsin and anti-plasmin activity. It rapidly hydrolyzes into non-active 4-guanidinobenzoic acid (GBA) and its active anti-proteolytic metabolite, 4-(4-guanidino-benzoyloxy) phenylacetic acid (GBPA, FOY-251). The mechanism by which Camostat Mesilate may have an effect on pancreatitis is not fully understood, but studies in rat models have shown Camostat Mesilate to suppress pro-inflammatory cytokine production, markedly decrease microscopic signs of inflammation, and suppress fibrosis associated with chronic pancreatitis. In one study it was observed that Camostat Mesilate decreased TGF-β1 concentrations and attenuated hepatic stellate cell activation as well as hepatic fibrosis, without entailing apparent systemic or local side effects. Other studies conducted in rats suggested that the administration of Camostat Mesilate leads to hypertrophy of pancreatic parenchyma and an increase in pancreas wet weight, as well as gross bodyweight. However, study results have been conflicting, as exemplified by another study that found no difference in body weight in response to treatment in dogs given various doses of Camostat Mesilate.

Camostat Mesilate has been approved for human use in Japan since 1985. The indication is “[T]o decrease acute symptoms in chronic pancreatitis” and is based on a series of clinical trials carried out in patients with pancreatitis where the indication of efficacy was gauged as a result of pain suppression, specifically abdominal pain.

The liver and kidneys are the predominant excretory organs for Camostat Mesilate and its metabolites, thus suggesting that serum biochemistry profiles may serve as a useful tool for assessing Camostat Mesilate toxicity in dogs. On a cellular level, acinar cell damage associated with chronic pancreatitis correlates with subsequent leakage of pancreatic lipase into the blood stream, the magnitude of which can be assessed by measurement of serum canine pancreatic lipase concentration (cPL). This canine biomarker can be measured by a specific, such as the Spec cPL® Test produced by IDEXX Laboratories, Westbrook, Me.). Under physiologic conditions canine pancreas-specific lipase in serum has a concentration of less than about 200 μg/L and concentrations of about 400 μg/L or more suggest a diagnosis of pancreatitis, leading to increased acinar permeability and leakage.

Anti-inflammatory properties have been attributed to Camostat Mesilate in a number of publications and canine specific assays, indicative of inflammation. C-reactive protein, S100A12, and trypsin-like immunoreactivity (cTLI) can be utilized to evaluate patients for non-specific inflammation and neutrophilic inflammation, respectively. C-reactive protein is an acute-phase protein synthesized by the liver and its concentration is increased above the reference interval in patients with systemic inflammation, regardless of the affected organ. In contrast to C-reactive protein, canine S100A12, is not a hepatic acute phase protein, but is secreted by neutrophilic granulocytes and thus reflects neutrophilic inflammation.

Transforming growth factor β1 (TGF-β1) is a multifunctional cytokine that plays a pivotal role in fibrosis in a number of parenchymal tissues. TGF-β1 is up-regulated along with its precursors during fibrosis secondary to chronic pancreatitis in humans and has been shown to be suppressed by Camostat Mesilate.

In view of the prevalence of pancreatitis in dogs and the lack of treatments a strong need exists for new treatment methodologies.

### SUMMARY OF THE INVENTION

New treatment methodologies for the treatment of canine pancreatitis are disclosed. The methods involve treating canine pancreatitis by first identifying whether a dog has pancreatitis, such as by measuring the serum concentration of canine pancreas-specific lipase and then administering a dose of Camostat Mesilate of between about 8 to about 200 mg/kg in 24 hrs (daily) when the dog has a serum concentration of about 400 μg/L or more of canine pancreas-specific lipase. A preferred dosage range is from about 24 to about 200 mg/kg in 24 hours. The drug can be given several times over the course of the day to reach the 24 hour dosage level. For example, the drug can be administered in 2, 3, or 4 administrations, as desired. Thus, from about 8 mg/kg to about 66.7 mg/kg can be administered every 8 hours to achieve the desired dosage amount of between 24 mg/kg/day to about 200 mg/kg. The drug can be conveniently administered in an oral capsule dosage form or by tablet. Treatment can be repeated on a daily basis for at least 7 days, more preferably a month (about 31 days), or several months or for as long as the treatment provides an improved quality of life.

A method for treating pancreatitis that involves identifying a patient with pancreatitis and administering to the patient a daily dose of Camostat Mesilate or 4-(4-guanidino-benzoyloxy) phenylacetic acid of between about 8 to about 1300 mg/kg or from between about 24 mg/kg to about 200 mg/kg is also disclosed. The dosage can be administered as a single dose or can be administered in 2, 3, 4 or more doses to achieve the daily dose. The treatment can be given for a week (7 days), a month (31 days) or several months, or for as long as the treatment provides an increased quality of life. The drug can be administered orally in an oral capsule.

### BRIEF DESCRIPTION OF DRAWINGS

FIG. 1 provides a time line of a clinical trial in which canine patients with pancreas-specific lipase of about 400 μg/L or more (as measured by Spec cPL®) were reevaluated 12±4 days after the initial sample submission to the gastrointestinal laboratory. Patients meeting pre-treatment requirements (cPL≧400 μg/L), serum triglyceride concentration<500 mg/dL, no major concurrent disease) received Camostat Mesilate for 21 to 31 days. This was followed by post-treatment evaluation.

FIG. 2 shows serum cPLI concentrations (as measured by Spec cPL®) in dogs with suspected chronic pancreatitis before and after treatment with two different dosages of Camostat Mesilate. Wilcoxon matched-pair signed rank tests were used to compare the values before and after treatment. There was no significant difference between pre-treatment (median: 501 μg/L; range: 344-6,052 μg/L) and post-treatment (median: 453 μg/L; range: 125-2,836 μg/L; n=12; p-value: 0.9697) cPLI concentrations of dogs treated with 4 mg/kg q 8 hrs. However, there was a significant difference between pre-treatment (median: 882 μg/L; range: 414 to 2,024 μg/L) and post-treatment (median: 633 μg/L; range: 193 to 2,580 μg/L; n=17; p-value: 0.0232) cPLI concentrations of dogs treated with 8 mg/kg q 8 hrs. Finally, there was no significant difference between pre-treatment (median: 690 μg/L; range: 344 to 6,052 μg/L) and post-treatment (median: 514 μg/L; range: 125 to 2,836 μg/L; n=29; p-value: 0.0903) cPLI concentrations for all dogs treated with Camostat Mesilate.

FIG. 3 shows serum cTLI concentrations in dogs with suspected chronic pancreatitis before and after treatment with two different dosages of Camostat Mesilate. Wilcoxon matched-pair signed rank tests were used to compare the values before and after treatment. There was no significant difference between pre-treatment (median: 22 μg/L; range: 20 to 92 μg/L) and post-treatment (median: 28 μg/L; range: 17 to 55 μg/L; n=11; p-value=0.8311) cTLI concentrations of dogs treated with 4 mg/kg q 8 hrs, between pre-treatment (median: 35 μg/L; range: 15 to 101 μg/L) and post-treatment (median: 22 μg/L; range: 6 to 81 μg/L; n=17; p-value=0.1594) cTLI concentrations of dogs treated with 8 mg/kg q 8 hrs and between pre-treatment (median: 32 μg/L; range: 15 to 101 μg/L) and post-treatment (median: 25 μg/L; range: 6 to 81 μg/L; n=28; p-value=0.2098) cTLI concentrations for all dogs treated with Camostat Mesilate.

FIG. 4 shows serum C-reactive protein concentrations in dogs with suspected chronic pancreatitis before and after treatment with two different dosages of Camostat Mesilate. Wilcoxon matched-pair signed rank tests were used to compare the values before and after treatment. There was no significant difference between pre-treatment (median: 0.8 mg/L; range: 0.1 to 30.3 mg/L) and post-treatment (median: 0.6 mg/L; range: 0.0 to 9.4 mg/L; n=12; p-value=0.1543) C-reactive protein concentrations of dogs treated with 4 mg/kg q 8 hrs, between pre-treatment (median: 0.1 mg/L; range: 0.1 to 16 mg/L) and post-treatment (median: 0.1 mg/L; range: 0.1 to 6.8 mg/L; n=17; p-value=0.25) C-reactive protein concentrations of dogs treated with 8 mg/kg q 8 hrs and between pre-treatment (median: 0.2 mg/L; range: 0.1 to 30.3 mg/L) and post-treatment (median: 0.1 mg/L; range: 0.0 to 9.4 mg/L; n=29; p-value=0.0595) C-reactive protein concentrations for all dogs treated with Camostat Mesilate.

FIG. 5 shows serum S100A12 concentrations in dogs with suspected chronic pancreatitis before and after treatment with two different dosages of Camostat Mesilate. Wilcoxon matched-pair signed rank tests were used to compare the values before and after treatment. There was a significant difference between pre-treatment (median: 160 μg/L; range: 52 to 1,327 μg/L) and post-treatment (median: 88 μg/L; range: 43 to 449 μg/L; n=11; p-value=0.0010) S100A12 concentrations of dogs treated with 4 mg/kg q 8 hrs. There was no significant difference between pre-treatment (median: 147 μg/L; range: 41 to 973 μg/L) and post-treatment (median: 148 μg/L; range: 58 to 746 μg/L; n=17; p-value=0.8176) S100A12 concentrations of dogs treated with 8 mg/kg q 8 hrs and between pre-treatment (median: 154 μg/L; range: 41 to 1,327 μg/L) and post-treatment (median: 133 μg/L; range: 43 to 746 μg/L; n=28; p-value=0.1438) S100A12 concentrations for all dogs treated with Camostat Mesilate.

FIG. 6 shows serum TGF-β1 concentrations in dogs with suspected chronic pancreatitis before and after treatment with two different dosages of Camostat Mesilate. Paired t-tests were used to compare the values before and after treatment. There was no significant difference between pre-treatment (mean: 45 ng/mL; SD: +13 ng/mL) and post-treatment (mean: 43 ng/mL; SD: ±8 ng/mL; n=12; p-value=0.3679) TGF-β1 concentrations of dogs treated with 4 mg/kg q 8 hrs. Also, there was no significant difference between pre-treatment (mean: 47 ng/mL; SD: ±20 ng/mL) and post-treatment (mean: 40 ng/mL; SD±12 ng/mL; n=17; p-value=0.1686) TGF-β1 concentrations of dogs treated with 8 mg/kg q 8 hrs and between pre-treatment (mean: 46 ng/mL; SD: ±17 ng/mL) and post-treatment (mean: 41 ng/mL; SD: ±11 ng/mL; n=29; p-value=0.0986) TGF-β1 concentrations for all dogs treated with Camostat Mesilate.

FIG. 7 shows serum cobalamin concentrations in dogs with suspected chronic pancreatitis before and after treatment with two different dosages of Camostat Mesilate. Paired t-tests and Wilcoxon matched-pair signed tests were used to compare the values before and after treatment. There was a significant difference between pre-treatment (mean: 868 ng/L; SD: ±108 ng/L) and post-treatment (mean: 695 ng/L; SD±223 ng/L; n=12; p-value=0.0363) cobalamin concentrations of dogs treated with 4 mg/kg q 8 hrs, as well as between pre-treatment (mean: 748 ng/L; SD: ±233 ng/L) and post-treatment (mean: 629 ng/L; SD: ±263 ng/L; n=17; p-value=0.0429) cobalamin concentrations of dogs treated with 8 mg/kg q 8 hrs. Finally, there was a significant difference between pre-treatment (median: 843 ng/L; range: 262 to 1001 ng/L) and post-treatment (median: 681 ng/L; range: 220 to 1001 ng/L; n=29; p-value=0.0007) cobalamin concentrations for all dogs treated with Camostat Mesilate.

### DETAILED DESCRIPTION OF THE INVENTION

The following abbreviations have the following definitions for purposes of this specification:

cPL canine pancreas-specific lipase

### CM Camostat Mesilate

cPLI canine pancreatic lipase immunoreactivity
cTLI canine trypsin-like immunoreactivity
ECG electrocardiogram
RI reference interval
Spec cPL canine pancreas-specific lipase

A method for treating pancreatitis is disclosed. The method involves first identifying a patient having pancreatitis. Any reliable method for diagnosing pancreatitis can be used. For example, pancreatitis has clinical symptoms that include increased vomiting, intermittent diarrhea, weight loss, anorexia or decreased appetite, lethargy, behavioral changes, abdominal discomfort and may be associated with diabetes mellitus and exocrine pancreatic insufficiency. Thus, in some instances pancreatitis can be diagnosed clinically. Other diagnostic methods that can be used for the diagnosis of pancreatitis include measurements of serum cTLI concentration, serum lipase activity, and abdominal ultrasound as are known. The preferred method for the diagnosis of pancreatitis in dogs is the serum cPLI concentration (pancreas-specific lipase concentrations). A serum cPLI concentration exceeding 400 μg/L in a dog is a strong predictor of canine pancreatitis and is the preferred method for identifying a patient for treatment with Camostat Mesilate.

Once a patient having pancreatitis is identified the patient can be treated by administering to the patient a therapeutically effective dose of Camostat Mesilate. A therapeutically effective dose can be determined functionally by improvements in the quality of life and observations of a reduction in clinical symptoms. Daily dosages of between 8 mg/kg to 1300 mg/kg can be administered to the patient. More preferably, daily dosages of about 100, 235, 550, 1300 mg/kg are contemplated. More preferably, the daily dosage will be in the range of about 24 mg/kg to about 200 mg/kg, particularly for dogs. For example, about 30, 40, 50, 60, 70, 80, 90, 100, 110, 120, 130, 140, 150, 160, 170, 180, or 190 mg of the drug can be administered per kg of a patient's mass per day. The word “about” when used in conjunction with a daily dosage means within 5 mg/kg.

Alternatively, the hydrolyzed active moiety of Camostat Mesilate, 4-(4-guanidino-benzoyloxy) phenylacetic acid, also known as GBPA and FOY-251 can be administered in an equivalent amount which is determined by the weight ratio of the two molecules.

Administration is preferably oral and can be conveniently administered within suitably sized capsules or tablets. The size of the capsule can vary depending on the amount of Camostat Mesilate being administered. For example, #2, #4, and #12 sized capsules can be used. Administration can be in a single daily dose or multiple doses. The drug can be administered in 2, 3, 4 or more doses as desired.

The drug can be administered as tablets. Suitable tablets can include 100 mg of Camostat Mesilate along with one or more excipients which can include hydroxypropyl cellulose, carmellose calcium, magnesium stearate, polyoxyethylene (105) polyoxypropylene (5) glycol and lactose hydrate. Preferred tablets contain all of the above excipients. When administered in capsule form one or more of the tablets can be included in the capsules and administered at the desired dosage. Any convenient amount of Camostat Mesilate can be included in a tablet or capsule. For example, 20, 40, 60, 80±10 mgs; 100, 150, 200, 250, 300, 350, 400, 450, 500, 550, 600, 650±25 mgs; 700, 800, 900, 1000, 1100±50 mgs, 1200, 1400, 1600, or 1800±100 mgs, etc. as desired.

A convenient dosing regimen can also be used that employs dosing based on banded weight ranges. Thus, for a target dose of 50 mg/kg/day a dose of 300 mg twice a day for dogs weighing up to 18 kg can be administered, a dose of 600 mg can be administered twice a day to dogs weighing between 19 and 33 kgs, a dose of 900 mg can be administered twice a day to dogs weighing between 34 to 45 kgs, and a dose of 1200 mgs can be administered twice a day to dogs weighing over 46 kgs.

Alternatively, dogs weighing between 10 to 40 pounds can be given 3 (100 mg) Camostat Mesylate tablets per day, dogs weighing between 40 to 73 pounds can be given 6 (100 mg) tablets per day, dogs weighing between 74 to 100 pounds can be given 9 (100 mg) tablets per day and dogs weighing more than 100 pounds can be given 12 (100 mg) tablets per day.

Another dosing regimen is provided in the Table below.

| | | | | | | Number of | | | |
|---|---|---|---|---|---|---|---|---|---|
| | | Daily | Total | Twice | Capsules | tablets | | Target | Total |
| Body | Body | Dose | Daily | daily | per | per | | Tabs | Tablets |
| weight | weight | (mg)/ | Dose | dose | Tablet | capsule | Capsule | per | Per |
| (kg) | (lb) | Kg | (mg) | CM | 100 mg | per dose | 250 | dose | Day |
| 1 | 2 | 50 | 50 | 25 | 100 | 0.25 | 250 | not | |
| | | | | | | | | eligible | |
| 2 | 4 | 50 | 100 | 50 | 100 | 0.50 | 250 | not | |
| | | | | | | | | eligible | |
| 3 | 7 | 50 | 150 | 75 | 100 | 0.75 | 250 | not | |
| | | | | | | | | eligible | |
| 4 | 9 | 50 | 200 | 100 | 100 | 1.00 | 250 | not | |
| | | | | | | | | eligible | |

Dogs weighing between 10 to 40 lbs can be given 6 tablets per day with
3 tablets per dose as shown below

| | | | | | | | | | |
|---|---|---|---|---|---|---|---|---|---|
| 5 | 11 | 50 | 250 | 125 | 100 | 1.25 | 250 | 3 | 6 |
| 6 | 13 | 50 | 300 | 150 | 100 | 1.50 | 250 | 3 | 6 |
| 7 | 15 | 50 | 350 | 175 | 100 | 1.75 | 250 | 3 | 6 |
| 8 | 18 | 50 | 400 | 200 | 100 | 2.00 | 250 | 3 | 6 |
| 9 | 20 | 50 | 450 | 225 | 100 | 2.25 | 250 | 3 | 6 |
| 10 | 22 | 50 | 500 | 250 | 100 | 2.50 | 250 | 3 | 6 |
| 11 | 24 | 50 | 550 | 275 | 100 | 2.75 | 250 | 3 | 6 |
| 12 | 26 | 50 | 600 | 300 | 100 | 3.00 | 250 | 3 | 6 |
| 13 | 29 | 50 | 650 | 325 | 100 | 3.25 | 250 | 3 | 6 |
| 14 | 31 | 50 | 700 | 350 | 100 | 3.50 | 250 | 3 | 6 |
| 15 | 33 | 50 | 750 | 375 | 100 | 3.75 | 250 | 3 | 6 |
| 16 | 35 | 50 | 800 | 400 | 100 | 4.00 | 250 | 3 | 6 |
| 17 | 37 | 50 | 850 | 425 | 100 | 4.25 | 250 | 3 | 6 |
| 18 | 40 | 50 | 900 | 450 | 100 | 4.50 | 250 | 3 | 6 |

Dogs weighing between 31 to 60 lbs can be given 12 tablets per day with
6 tablets per dose as shown below

| | | | | | | | | | |
|---|---|---|---|---|---|---|---|---|---|
| 19 | 42 | 50 | 950 | 475 | 100 | 4.75 | 250 | 6 | 12 |
| 20 | 44 | 50 | 1000 | 500 | 100 | 5.00 | 250 | 6 | 12 |
| 21 | 46 | 50 | 1050 | 525 | 100 | 5.25 | 250 | 6 | 12 |
| 22 | 48 | 50 | 1100 | 550 | 100 | 5.50 | 250 | 6 | 12 |
| 23 | 51 | 50 | 1150 | 575 | 100 | 5.75 | 250 | 6 | 12 |
| 24 | 53 | 50 | 1200 | 600 | 100 | 6.00 | 250 | 6 | 12 |
| 25 | 55 | 50 | 1250 | 625 | 100 | 6.25 | 250 | 6 | 12 |
| 26 | 57 | 50 | 1300 | 650 | 100 | 6.50 | 250 | 6 | 12 |
| 27 | 59 | 50 | 1350 | 675 | 100 | 6.75 | 250 | 6 | 12 |
| 28 | 62 | 50 | 1400 | 700 | 100 | 7.00 | 250 | 6 | 12 |
| 29 | 64 | 50 | 1450 | 725 | 100 | 7.25 | 250 | 6 | 12 |
| 30 | 66 | 50 | 1500 | 750 | 100 | 7.50 | 250 | 6 | 12 |
| 31 | 68 | 50 | 1550 | 775 | 100 | 7.75 | 250 | 6 | 12 |
| 32 | 70 | 50 | 1600 | 800 | 100 | 8.00 | 250 | 6 | 12 |
| 33 | 73 | 50 | 1650 | 825 | 100 | 8.25 | 250 | 6 | 12 |

Dogs weighing between 61 to 85 lbs can be given 18 tablets per day with
9 tablets per dose as shown below

| | | | | | | | | | |
|---|---|---|---|---|---|---|---|---|---|
| 34 | 75 | 50 | 1700 | 850 | 100 | 8.50 | 250 | 9 | 18 |
| 35 | 77 | 50 | 1750 | 875 | 100 | 8.75 | 250 | 9 | 18 |
| 36 | 79 | 50 | 1800 | 900 | 100 | 9.00 | 250 | 9 | 18 |
| 37 | 81 | 50 | 1850 | 925 | 100 | 9.25 | 250 | 9 | 18 |
| 38 | 84 | 50 | 1900 | 950 | 100 | 9.50 | 250 | 9 | 18 |
| 39 | 86 | 50 | 1950 | 975 | 100 | 9.75 | 250 | 9 | 18 |
| 40 | 88 | 50 | 2000 | 1000 | 100 | 10.00 | 250 | 9 | 18 |
| 41 | 90 | 50 | 2050 | 1025 | 100 | 10.25 | | 9 | 18 |
| 42 | 92 | 50 | 2100 | 1050 | 100 | 10.50 | | 9 | 18 |
| 43 | 95 | 50 | 2150 | 1075 | 100 | 10.75 | | 9 | 18 |
| 44 | 97 | 50 | 2200 | 1100 | 100 | 11.00 | | 9 | 18 |
| 45 | 99 | 50 | 2250 | 1125 | 100 | 11.25 | | 9 | 18 |

Possible dosing for dogs weighing more than 100 lbs

| | | | | | | | |
|---|---|---|---|---|---|---|---|
| 46 | 101 | 50 | 2300 | 1150 | 100 | 11.50 | 12 |
| 47 | 103 | 50 | 2350 | 1175 | 100 | 11.75 | 12 |
| 48 | 106 | 50 | 2400 | 1200 | 100 | 12.00 | 12 |
| 49 | 108 | 50 | 2450 | 1225 | 100 | 12.25 | 12 |
| 50 | 110 | 50 | 2500 | 1250 | 100 | 12.50 | 12 |
| 51 | 112 | 50 | 2550 | 1275 | 100 | 12.75 | 12 |
| 52 | 114 | 50 | 2600 | 1300 | 100 | 13.00 | 12 |
| 53 | 117 | 50 | 2650 | 1325 | 100 | 13.25 | 12 |
| 54 | 119 | 50 | 2700 | 1350 | 100 | 13.50 | 12 |
| 55 | 121 | 50 | 2750 | 1375 | 100 | 13.75 | 12 |
| 56 | 123 | 50 | 2800 | 1400 | 100 | 14.00 | 12 |
| 57 | 125 | 50 | 2850 | 1425 | 100 | 14.25 | 12 |
| 58 | 128 | 50 | 2900 | 1450 | 100 | 14.50 | 12 |
| 59 | 130 | 50 | 2950 | 1475 | 100 | 14.75 | 15 |
| 60 | 132 | 50 | 3000 | 1500 | 100 | 15.00 | 15 |
| 61 | 134 | 50 | 3050 | 1525 | 100 | 15.25 | 15 |
| 62 | 136 | 50 | 3100 | 1550 | 100 | 15.50 | 15 |
| 63 | 139 | 50 | 3150 | 1575 | 100 | 15.75 | 15 |

Treatment can be for as long as the patient's quality of life is improved. As used herein “improved quality of life” means a reduction in observed clinical symptoms and/or a concentration of cPLI in the serum of below 400 μg/L. The treatment will generally last for at least 7 days but longer treatments lasting a month or several months are contemplated. For example, treatments can past for about 180 days or longer.

The disclosed methods are considered especially useful in the treatment of dogs which appear to be particularly vulnerable to pancreatitis. Therefore, a method for treating canine pancreatitis is specifically contemplated. The method can include measuring the serum concentration of canine pancreas-specific lipase as described herein and then administering a dose of Camostat Mesilate of from about 8 to about 200 mg/kg in 24 hrs when the dog has a serum concentration of canine pancreas-specific lipase of about 400 μg/L or more.

In a dog the preferred treatment is with a daily dose of Camostat Mesilate of from about 24 to about 200 mg/kg with administration from one to three times daily at the discretion of the treating veterinarian and the daily treatment repeated for at least 7 days. Generally, for any patient when multiple administrations are employed it is preferable to give them in approximately even intervals throughout the day. Thus, when 3 doses are given per day they are given about every 8 hours. About 8 hours in this instance means within 30 minutes of 8 hours. The treatment period can be from 7 days to 31 days (months) or for as long as the dog's quality of life is improved by the treatments.

### Example 1

The administration of two different dosages (4 mg/kg q 8 hrs and 8 mg/kg q 8 hrs) of Camostat Mesilate to 29 dogs with suspected canine pancreatitis is disclosed followed by an assessment of the therapeutic impact of the treatment on acinar cell damage, pancreatic function, and systemic markers of inflammation and fibrosis. Treatment effects on overall health status, quality of life, and clinical signs were also evaluated with serum biochemistry profiles, complete blood counts, and owner questionnaires.

### Sample Population

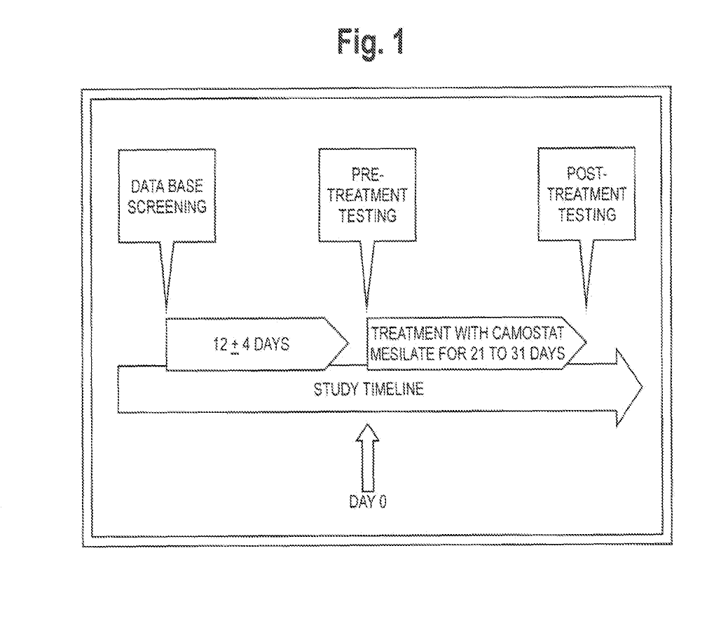

Dogs were identified by screening current submissions to the Gastrointestinal Laboratory at Texas A&M indicative of pancreatitis (canine pancreas-specific lipase≧400 μg/L) using an immunoassay for measurement of canine pancreas-specific lipase in canine serum known as the spec CPL® Test (IDEXX, Laboratories). A number of potential study participants were directly referred to the investigator by veterinarians, based on positive pet-side SNAP cPL® Test (IDEXX Laboratories, Westbrook, Me., USA) results and concomitant clinical signs. After ascertaining individual suitability of a patient with the referring veterinarian, serum canine pancreas-specific lipase concentration, serum biochemistry profile, and complete blood count were evaluated or re-evaluated 12±4 days after collection of the initial sample submission to the Gastrointestinal Laboratory. Based on the assumption that consecutive canine pancreas-specific lipase elevations indicate chronic pancreatitis, enrollment was offered for patients with canine pancreas-specific lipase concentrations≧400 μg/L, serum triglyceride concentrations below 500 mg/dL, and no indication of hepatic, renal, or other organ disease. As shown in FIG. 1, on average 3 to 5 days passed between pre-treatment testing and initiation of treatment (FIG. 1). Owner consent was required prior to phlebotomy and for enrollment into the trial.

### Treatment and Owner Instructions

The Camostat Mesilate dosage was determined based on dosages reported to be safe in a sub-acute toxicity studies in dogs. Twelve enrolled dogs received camostat capsules at a dosage of 4 mg/kg q 8 hrs, for a total dose of 12 mg/kg q 24 hrs. The remaining 17 dogs received a dosage of 8 mg/kg q 8 hrs of Camostat Mesilate in capsules, for a total of 24 mg/kg q 24 hrs. Individual patients were medicated for between 21 to 31 days. Camostat Mesilate capsules used for this trial were compounded with hard gelatin capsules in sizes ranging from #2, #3, and #4 (depending of the body weight of the dogs) from Capsugel, Morristown, N.J., USA.

Patients were treated at home and their overall health was monitored by their owners and the referring physician.

Owners of enrolled dogs received instructions to refrain from changing the dog's diet during the trial period and to report on the overall health status and quality of life of their pet.

### Sample Analysis

Blood samples were collected before beginning the treatment and after completion of the treatment period. Complete blood count (red blood cell count, hematocrit, mean corpuscular volume, mean corpuscular hemoglobin, mean corpuscular hemoglobin concentration, differential and absolute white blood cell count, red blood cell count, hemoglobin, platelet estimate, red blood cell morphology, white blood cell morphology, and hemoparasites) was evaluated at the Texas Veterinary Medical Diagnostic Laboratory (TVMDL). A serum chemistry profile with electrolytes (glucose, blood urea nitrogen, creatinine, calcium, phosphorus, albumin, total protein, total bilirubin, alkaline phosphatase, alanine amino transferase, aspartate amino transferase, γ-glutamyl transferase, cholesterol, triglycerides, globuline, sodium, potassium, and chloride) was performed on a SIRRUS analyzer (Stanbio, Boerne, Tex., USA). Serum concentrations of cPL (RI: 0-200 μg/L; Spec cPL® IDEXX Laboratories, Westbrook, Me., USA), cTLI (RI: 5.7-45.2 μg/L; Canine TLI DOUBLE ANTIBODY®, Siemens Healthcare Diagnostics, Los Angeles, Calif., USA), cobalamin (RI: 251-908 ng/L), and folate (RI: 7.7-24.4 μg/L; Immulite 2000®, Siemens Healthcare Diagnostics Inc., Deerfield, Ill., USA), canine specific C-reactive protein (RI: 0-7.6 mg/L, Tridelta Development Limited, Maynooth, CO. Kildare, Ireland), S100A12 (RI: 56-331 μg/L, Gastrointestinal Laboratory in-house radioimmunoassay), and TGF-β1 (RI: 38-72 ng/mL; Quantikine® ELISA, R&D Systems, Minneapolis, Nebr., USA) were performed at the Gastrointestinal Lab at Texas A&M University.

Additionally, samples that exceeded the working range of the Spec cPL assay were diluted and re-analyzed.

All serum samples were refrigerated and shipped overnight on ice packs and were stored at −20° C. until final cPLI, C-reactive protein, S100A12, and TGF-β1 testing.

Pre-treatment and post-treatment results for all diagnostic tests were statistically analyzed using Prism 6 (GraphPad Software, La Jolla, Calif., USA). The D'Agostino-Pearson omnibus test was used to evaluate all datasets for Gaussian distribution. Groups were compared using a paired t-test for normally-distributed data sets and a Wilcoxon matched-pairs test when Gaussian distribution could not be assumed. Mean values and standard deviation are reported for data sets with Gaussian distribution. Median and minimum/maximum values are reported for data sets where Gaussian distribution could not be assumed. The significance threshold was set at p=0.05.

### Results

### Signalment

The treatment group treated with 4 mg/kg q 8 hrs Camostat Mesilate consisted of 1 intact female, 7 spayed females, and 4 neutered males with an average age [range] of 10.5 [2-14] years. The group treated with 8 mg/kg q 8 hrs Camostat Mesilate consisted of 4 intact females, 8 spayed females and 5 neutered males with an average age [range] of 11.5 [3-15] years. Overall, 20/29 dogs (70%) were 10 years or older and 15/29 dogs (52%) were spayed females. Dachshunds (n=6) and mixed breed dogs (n=6), followed by Miniature Schnauzers (n=3) and Cavalier King Charles Spaniels (n=2) were the most prominent breeds.

### Adverse Drug Reactions

Two dogs treated with 4 mg/kg q 8 hrs were reported to have increased flatulence.

One dog treated with 8 mg/kg q 8 hrs exhibited signs of pruritus in one front paw and another dog was reported to have increased flatulence. No other clinical signs suggestive of an adverse drug reaction were observed in either group.

### Drop-Outs

Five of 34 (15%) initially enrolled patients were unavailable for post-treatment follow up. Three had been euthanized due to pre-existing conditions unrelated to the study (e.g., advanced neoplasia). These conditions had been diagnosed after the patients had entered the study and no indication thereof was found during pre-treatment workup. One patient owner chose to abort the study on account of personal preferences, and one patient owner scheduled the post-treatment appointment with the participating veterinarian two weeks after they had stopped medicating the patient, rendering the collected sample useless for statistical analysis.

### Complete Blood Count and Serum Biochemistry Profile

There were no statistically significant differences between pre- and post-treatment complete blood count and serum biochemistry parameters. However, one dog showed a 35-fold increase in serum γ-glutamyl transferase activity and a 6-fold increase in aspartate amino transferase activity after treatment. Another dog showed a 6-fold increase in serum aspartate amino transferase activity. No follow-up data is available to investigate whether or not these findings normalized.

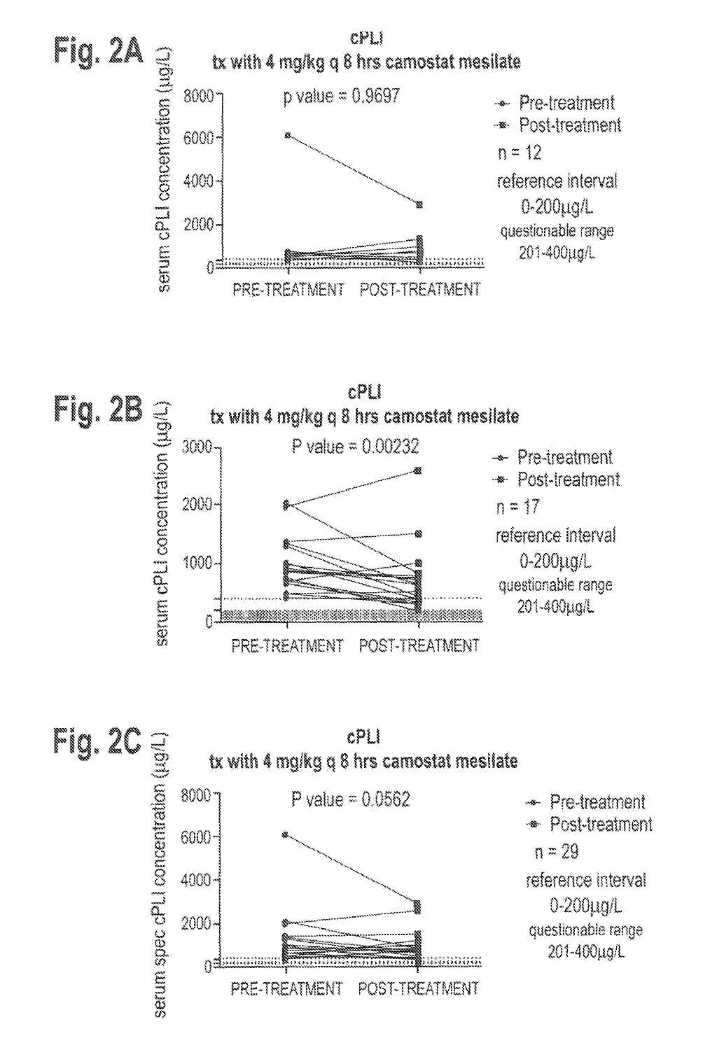

### Serum Pancreas-Specific Lipase Concentrations (FIG. 2)

Serum cPL concentrations did not change significantly between pre-treatment (median: 501 μg/L; range: 344 to 6,052 μg/L) and post-treatment (median: 453 μg/L; range: 125 to 2,836 μg/L) values in the 4 mg/kg q 8 hrs group (n=12; p=0.9697). However, a statistically significant change was observed between pre-treatment (median: 882 μg/L; range: 414 to 2,024 μg/L) and post-treatment (median: 633; range: 193 to 2,580 μg/L) in the 8 mg/kg q 8 hrs group (n=17; p=0.0232). Also, while there was a trend towards a decrease in serum cPL concentrations post-treatment, there was no statistically significant difference between pre-treatment (median: 690 μg/L; range: 344 to 6,052 μg/L) and post-treatment (median: 514 μg/L; range: 125 to 2,836 μg/L) for all treated dogs combined (n=29; p=0.0562). This last statistic includes all 4 mg/kg and all 8 mg/kg data points.

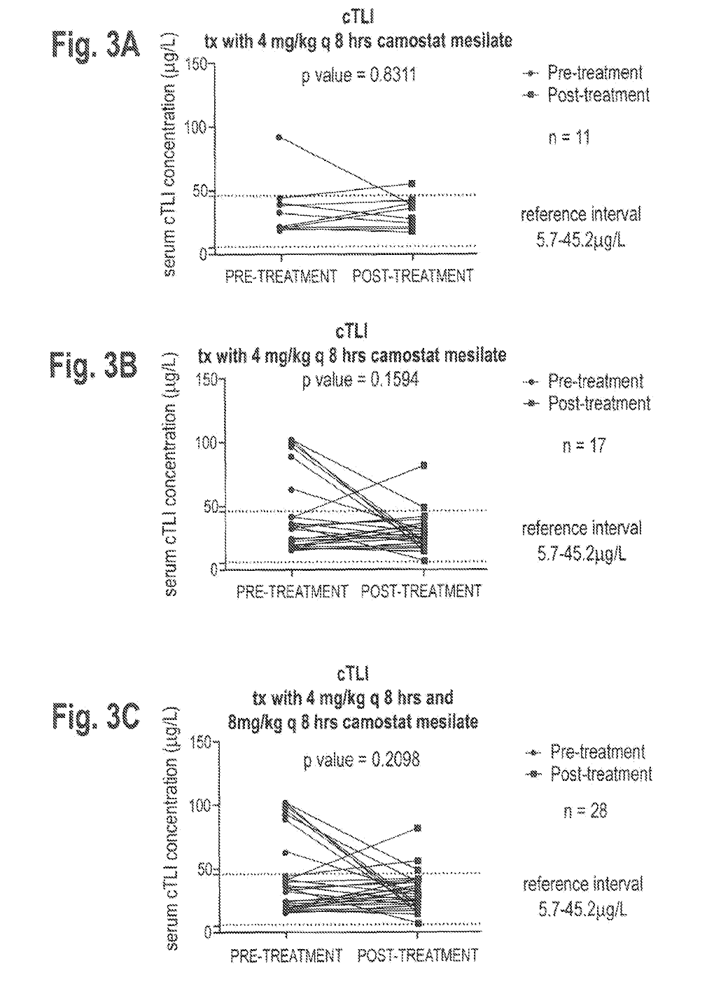

Serum cTLI Concentration (FIG. 3)

cTLI concentrations did not change significantly between pre-treatment (median: 22 μg/L; range: 20 to 92 μg/L) and post-treatment (median: 28 μg/L; range: 17 to 55 μg/L) values in the 4 mg/kg q 8 hrs group (n=11; p=0.8311). No significant changes in cTLI concentrations were observed between pre-treatment (median: 35 μg/L; range: 15 to 101 μg/L) and post-treatment (median: 24 μg/L; range: 15 to 101 μg/L) values in the 8 mg/kg q 8 hrs group (n=17; p=0.1594) and between pre-treatment (median: 32 μg/L; range: 15 to 101 μg/L) and post-treatment (median: 25 μg/L; range: 6 to 81 μg/L) values for all treated dogs combined (n=28; p=0.2098).

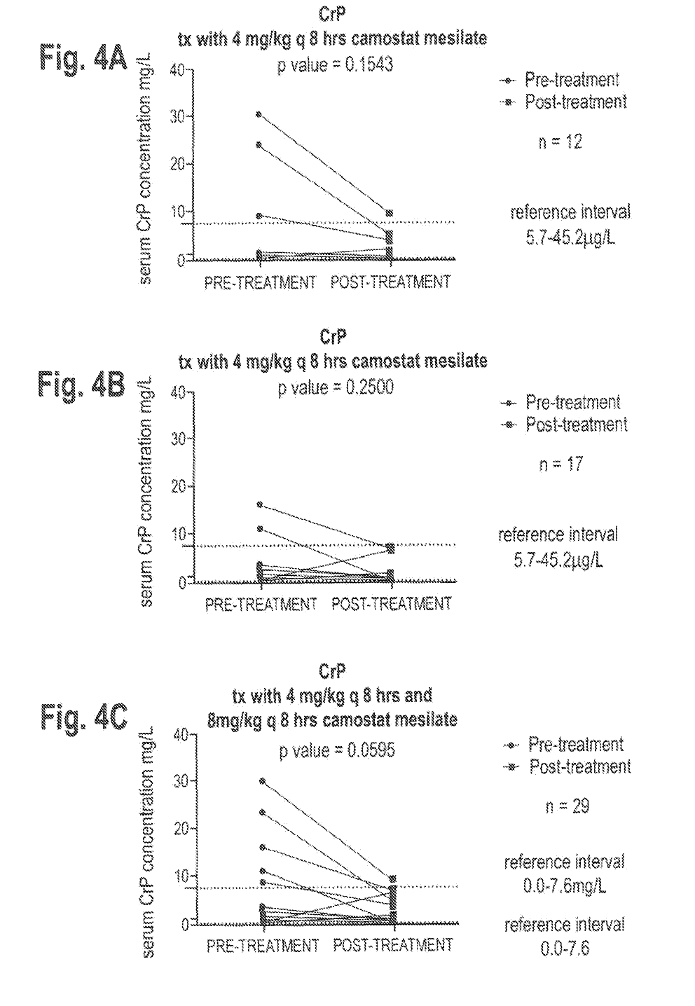

### Serum C-Reactive Protein Concentrations (FIG. 4)

C-reactive protein concentrations did not change significantly between pre-treatment (median: 0.8 mg/L; range: 0.1 to 30.3 mg/L) and post-treatment (median: 0.6 mg/L; range: 0.0 to 9.4 mg/L) values in the 4 mg/kg q 8 hrs group (n=12; p=0.1543), between pre-treatment (median: 0.1 mg/L; range: 0.1 to 16.0 mg/L) and post-treatment (median: 0.1 mg/L; range: 0.1 to 6.8 mg/L) values in the 8 mg/kg q 8 hrs group (n=17; p=0.2500). Also, while there was a trend towards a decrease in serum C-reactive protein concentrations post-treatment, there was no statistically significant difference between pre-treatment (median: 0.2 mg/L; range: 0.1 to 30.3 mg/L) and post-treatment (median: 0.1 mg/L; range: 0.1 to 9.4 mg/L) for all treated dogs combined (n=29; p=0.0595).

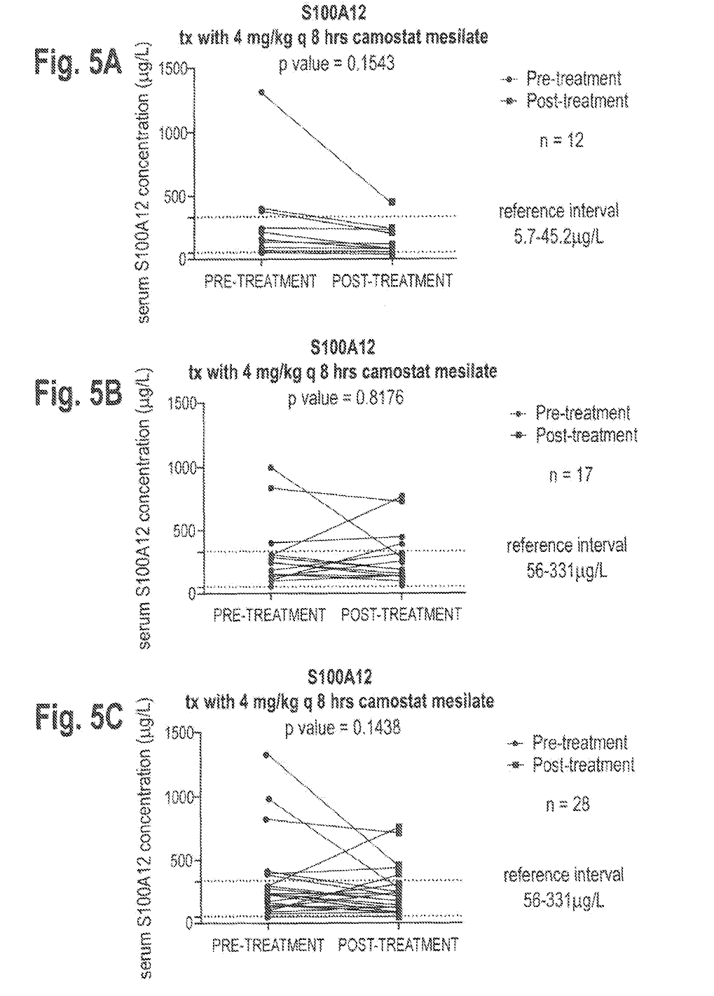

### Serum S100A12 Concentrations (FIG. 5)

Serum S100A12 concentrations decreased significantly between pre-treatment (median: 160 μg/L; range: 52 to 1327 μg/L) and post-treatment (median: 88 μg/L; range: 43 to 449 μg/L) values in the 4 mg/kg q 8 hrs group (n=11; p=0.0010). Serum S100A12 concentrations did not change significantly between pre-treatment (median: 147 μg/L; range: 41 to 973 μg/L) and post-treatment (median: 148 μg/L; range: 58 to 746 μg/L) values in the 8 mg/kg q 8 hrs group (n=17; p=0.8176) and between pre-treatment (median: 154 μg/L; range: 41 to 1327 μg/L) and post-treatment (median: 133 μg/L; range: 43 to 746 μg/L) values for all treated dogs combined (n=28; p=0.1438).

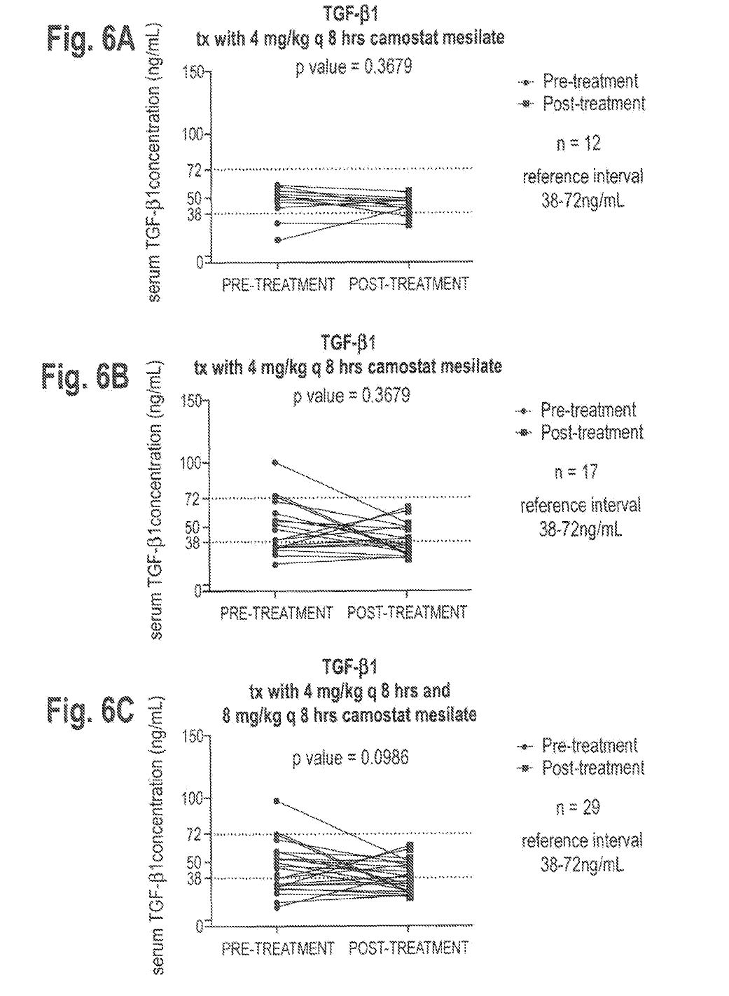

### Serum TGF-β1 Concentrations (FIG. 6)

Serum TGF-β1 concentrations did not change significantly between pre-treatment (mean: 45 ng/mL; SD: ±13 ng/mL) and post-treatment (mean: 43 ng/mL, SD: ±8 ng/mL) values in the 4 mg/kg q 8 hrs group (n=12; p=0.3679) and between pre-treatment (mean: 47 ng/mL; SD: ±20 ng/mL) and post-treatment (mean: 40 ng/mL; SD: ±12 ng/mL) values in the 8 mg/kg q 8 hrs group (n=17; p=0.1686). Also, while there was a trend towards a decrease in serum TGF-β1 concentrations post-treatment, there was no statistically significant difference between pre-treatment (mean: 46 ng/mL; SD: ±17 ng/mL) and post-treatment (mean: 41 ng/mL; SD: ±11 ng/mL) values for all treated dogs combined (n=29; p=0.0986).

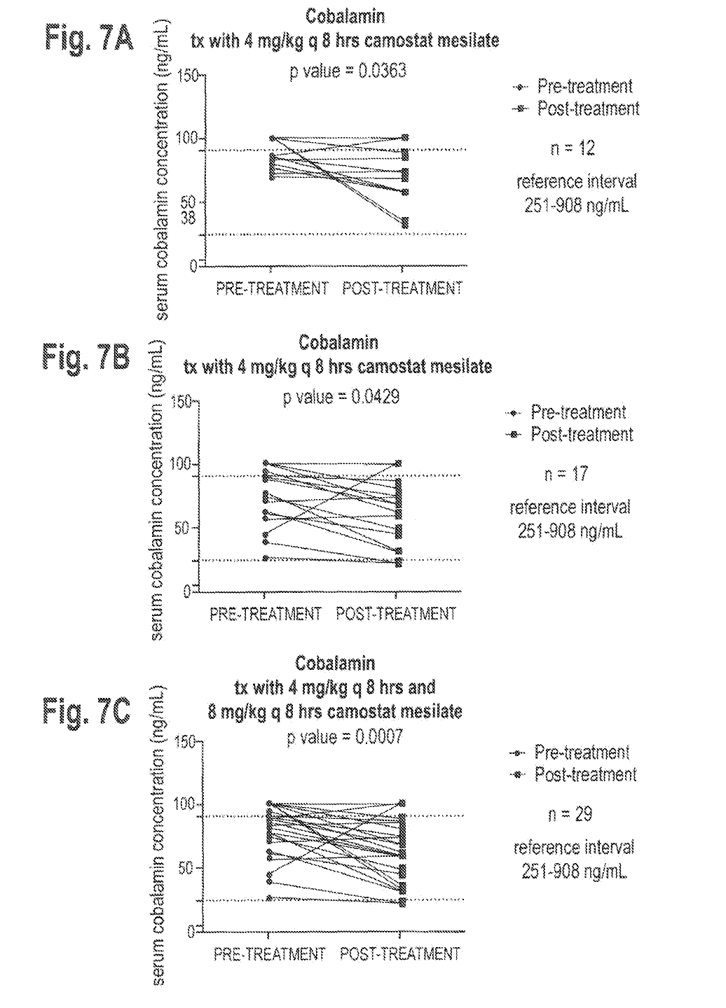

### Serum Cobalamin Concentrations (FIG. 7)

Serum cobalamin concentrations decreased significantly between pre-treatment (mean: 868 ng/L; SD: +108 ng/L) and post-treatment (mean: 695 ng/L; SD: +223 ng/L) values in the 4 mg/kg q 8 hrs group (n=12; p=0.0363), between pre-treatment (mean: 748 ng/L; SD: +233 ng/L) and post-treatment (mean: 629 ng/L; SD: ±263 ng/L) values in the 8 mg/kg q 8 hrs group (n=17; p=0.0429) and between pre-treatment (median: 843 ng/L; range: 262 to 1001 ng/L) and post-treatment (median: 681 ng/L; range: 220 to 1001 ng/L) values for all treated dogs combined (n=29; p=0.0007).

### Serum Folate

No changes in serum folate concentration between pre-treatment and post-treatment values in response to treatment were observed for either group (data not shown).

### Discussion

The Camostat Mesilate dosage of 4 mg/kg q 8 hrs chosen at the beginning of the study reflected a cautious choice, based on doses found safe in a previously published sub-acute toxicity study and it was deemed prudent to only increase dosage to 8 mg/kg q 8 hrs after it was ascertained that Camostat Mesilate did not elicit any adverse drug effects in the earlier treatment group.

Surprisingly, serum cobalamin concentrations decreased significantly in both treatment groups during the treatment with Camostat Mesilate. However, even more surprisingly, 13 of the 29 study patients had been on cobalamin supplementation prior to enrollment into the study. This may have been due to diagnosed or suspected concurrent small intestinal disease of these patients prior to enrollment into the study. Continuing cobalamin supplementation was not part of the treatment protocol, which may explain why serum cobalamin concentrations decreased significantly. However, further studies will be required to determine any effect of Camostat Mesilate on cobalamin homeostatis and serum cobalamin concentrations.

Only 1 patient in the 4 mg/kg and 6 patients in the 8 mg/kg treatment groups had an increased pre-treatment serum cTLI concentration. This supports the finding that only 30 to 40% of dogs with pancreatitis have increased serum cTLI concentrations and serum cTLI concentration is an inferior test for the diagnosis of pancreatitis in dogs. Also, the number of patients with an increased serum cTLI concentration prior to treatment is too low to arrive at a conclusion as to whether Camostat Mesilate has any impact on cTLI concentration as an indicator for pancreatic function.

The overall consensus from participating pet owners was that their dog's quality of life had improved.

### Conclusion

Twenty-nine dogs with suspected chronic pancreatitis based on clinical signs and repeated measurements of an increased serum concentration of canine pancreas-specific lipase above a cut-off value for diagnosis of pancreatitis of about 400 μg/L or more received a dose of 4 or 8 mg/kg Camostat Mesilate q 8 hrs (12 or 24 mg/kg q d) over a period of 26±5 days.

Treatment of dogs with both dosages of Camostat Mesilate was only associated with very mild side effects, suggesting that this drug is generally safe for use in dogs. Also Camostat Mesilate administered orally at a dose of 24 mg/kg q d, led to a subjective improvement of quality of life in some patients based on reports from referring vets and pet owners. In addition, dogs in this study given an oral daily dose of 24 mg/kg showed a statistically significant decrease in serum concentrations of canine pancreas-specific lipase indicating attenuation of acinar cell damage. However, Camostat Mesilate did not lead to any significant changes in serum concentrations of C-reactive protein, S100A12, or TGF-β1, even though marked decreases were observed in individual patients.

### Example 2

### Rising-Dose and Multiple-Dose Tolerance Studies of Camostat Mesilate Using Oral Capsule Administration in Dogs

The present example determines the toxicity of Camostat Mesilate and the upper dosage limit of Camostat Mesilate in dogs. Camostat Mesilate was given orally by capsule as a single dose to dogs followed by at least a 3-day washout period repeated for up to 5 cycles. In addition, the potential toxicity of Camostat Mesilate was determined when given orally 3 times a day by capsule for 14 days to dogs.

The study design was as follows:

TABLE 1
Experimental Design For The Rising-Dose Phase

| | | | | No. of Animals | |
|---|---|---|---|---|---|
| Group | | | Dose Level | Rising-dose Study | |
| No. | Test Material | Cycle | (mg/kg) | Males | Females |
| 1 | Camostat Mesilate | 1 | 10 | 1 | 1 |
| | Camostat Mesilate | 2 | 30 | | |
| | Camostat Mesilate | 3 | 100 | | |
| | Camostat Mesilate | 4 | 200 | | |
| | Camostat Mesilate | 5 | 250 | | |

The following parameters and end points were evaluated in the rising-dose phase of this study: clinical signs, body weights, body weight changes, food consumption, electrocardiography, clinical pathology parameters (hematology, clinical chemistry, and urinalysis), and gross necropsy findings.

TABLE 2
Experimental Design For The Multiple-Dose Phase

| | | | | No. of Animals | |
|---|---|---|---|---|---|
| Group | | Dose Level | Dose Level | Multiple-dose Study | |
| No. | Test Material | (mg/kg/day) | (mg/kg/dose) | Males | Females |
| 1 | Camostat | 200 | 66.7 | 1 | 1 |
| | Mesilate | | | | |

The following parameters and end points were evaluated in the multiple-dose phase of this study: clinical signs, body weights, body weight changes, food consumption, electrocardiography, clinical pathology parameters (hematology, clinical chemistry, and urinalysis), and gross necropsy findings.

Rising-Dose Phase:

All animals survived to scheduled euthanasia. The most notable clinical observations were salivation and soft feces/feces with mucoid material at 200 and 250 mg/kg. Following each dose level, there were no notable changes in body weights, food consumption, hematology parameters, or urinalysis parameters. There were no toxicologic effects on ECG rhythm or measurements. Potential test article-related changes in clinical chemistry parameters were limited to increased creatinine, total bilirubin, and urea nitrogen in the male following the 250 mg/kg dose and increased creatine kinase in the female following the 200 and 250 mg/kg doses. There were no gross necropsy findings. Based on these results, 200 mg/kg was tested in the multiple-dose phase.

Multiple-Dose Phase:

All animals survived to scheduled euthanasia. In the male, clinical observations were limited to thin appearance and a single occurrence of vomitus. In the female, clinical observations were limited to clear ocular discharge and single occurrences of vomitus and mucoid feces. Body weight loss was observed in both the male (−9.2%) and female (−2.7%). There were no notable changes in food consumption, hematology parameters, or urinalysis parameters. There were no toxicologic effects on ECG rhythm or measurements. Potential test article-related clinical chemistry parameters were limited to increased creatinine and γ-glutamyl transferase in the male and female. There were no gross necropsy findings.

Oral administration of Camostat Mesilate 3 times per day at 200 mg/kg/day for 14 days resulted in body weight loss and potential test article-related increases in creatinine and γ-glutamyl transferase. Based on these results, 200 mg/kg/day will be chosen as the high-dose level.

### Materials and Methods

### Test Article

Identification: Camostat Mesilate
Physical Description: White powder
Purity: 100% (assumed for calculation purposes, dose calculations were not corrected for purity)
Storage Conditions: Kept in a refrigerator set to maintain 5° C.
Supplier: Stason Pharmaceuticals

### Control Article

Identification: Empty Capsule, size #12
Physical Description: Transparent capsule
Storage Conditions: Kept in a room temperature area

### Reserve Samples

For each batch (lot) of test and control article, a reserve sample (1 g/l empty capsule) was collected and maintained under the appropriate storage.

### Dose Formulation and Analysis

### Preparation of Test Article

The appropriate amount of test article, Camostat Mesilate, for each animal was weighed and placed into a gelatin capsule or capsules (equally divided) based on the most recent scheduled body weight. The dosing capsules were prepared on the day of dosing for the rising-dose phase and at least twice weekly for the multiple-dose phase, placed into individually labeled pill bottles sufficient for each day of dosing, placed in plastic bags, stored in a refrigerator set to maintain 5° C. until use (unless maintained at room temperature for dosing on the same day), and dispensed daily. On each day of dosing, the bag containing the capsules that were maintained refrigerated was taken out of the refrigerator and left out for at least 30 minutes at room temperature before opening and dosing.

Details of the preparation and dispensing of the test article have been retained in the Study Records.

### Test System

3 male and 3 female Beagle dogs were received from Marshall BioResources, North Rose, N.Y. The animals were examined and weighed on the day following receipt. The rising-dose phase animals were 6 months old and weighed 7.5 kg and 8.3 kg at initiation of dosing. The multiple-dose phase animals were 7 months old and weighed 7.4 kg and 7.6 kg at initiation of dosing.

The Beagle dog was chosen as the animal model for this study because it is a preferred non-rodent species for preclinical toxicity testing by regulatory agencies. The total number of animals used in this study was considered to be the minimum required to properly characterize the effects of the test article and has been designed such that it does not require an unnecessary number of animals to accomplish its objectives.

The animals were acclimated to their designated housing for at least 10 days before the first day of dosing. Animals assigned to study were assigned to groups in a random order. Animals in poor health or at extremes of body weight range were not assigned to groups.

Animals were housed individually throughout the study in suspended stainless steel cages equipped with an automatic watering valve. Housing and care were as specified in the USDA Animal Welfare Act (9 CFR, Parts 1, 2, and 3) and as described in the Guide for the Care and Use of Laboratory Animals from the National Research Council.

Temperatures of 64° F. to 71° F. (18° C. to 22° C.) with a relative humidity of 46% to 62% were maintained. A 12-hour light/12-hour dark cycle was maintained, except when interrupted for designated procedures. Ten or greater air changes per hour with 100% fresh air (no air recirculation) were maintained in the animal rooms.

PMI Nutrition International Certified Canine Chow No. 5007 (25% protein) was provided as a daily ration throughout the study. An approximate 300 g ration of feed was provided once daily, except during designated procedures, to each dog beginning on the day after receipt. The food ration was offered up to 2 hours after the first daily dosing. The feed was left in the dog's cage for a period of 2 to 4 hours and then removed. The lot number and expiration date of each batch of diet used during the study were recorded. The feed was analyzed by the supplier for nutritional components and environmental contaminants. Based on these results, there were no contaminants that would interfere with the conduct or interpretation of the study.

Municipal tap water, after treatment by reverse osmosis and ultraviolet irradiation, was freely available to each animal via an automatic watering system (except during designated procedures). Periodic analysis of the water is performed. There were no known contaminants in the water that could interfere with the outcome of the study.

Beginning at receipt, an animal enrichment device (Hercules bone) was provided to each animal. As a reward and means to promote operant conditioning and desired behavior, each animal was offered a certified treat at least 3 times per week.

The animals were moved to a designated exercise area within the animal room a minimum of 2 times per week for exercise/socialization. Care was taken to ensure that males and females were exercised/socialized separately by sex and treatment group. Veterinary care was available throughout the course of the study; however, no treatments were required.

### Experimental Design

TABLE 3
Experimental Design For The Rising-Dose Phase

| | | | | No. of Animals | |
|---|---|---|---|---|---|
| Group | | | Dose Level | Rising-dose Study | |
| No. | Test Material | Cycle | (mg/kg) | Males | Females |
| 1 | Camostat Mesilate | 1 | 10 | 1 | 1 |
| | Camostat Mesilate | 2 | 30 | | |
| | Camostat Mesilate | 3 | 100 | | |
| | Camostat Mesilate | 4 | 200 | | |
| | Camostat Mesilate | 5 | 250 | | |

TABLE 4
Experimental Design for the Multiple-dose Phase

| | | | | No. of Animals | |
|---|---|---|---|---|---|
| Group | | Dose Level | Dose Level | Multiple-dose Study | |
| No. | Test Material | (mg/kg/day) | (mg/kg/dose) | Males | Females |
| 1 | Camostat | 200 | 66.7 | 1 | 1 |
| | Mesilate | | | | |

For the rising-dose phase, the test article was administered to the appropriate animals by oral capsule. At least a 3-day washout period was maintained before proceeding to the next dose level; therefore, dosing occurred on Days 1, 5, 9, 15, and 19. The dose amount for each animal was based on the most recent body weight measurement. The first day of dosing was designated as Study Day 1.

For the multiple-dose phase, the test article was administered to the appropriate animals 3 times a day in an oral capsule from Days 1 to 14. The doses were administered 8 hours±30 minutes apart. The dose amount for each animal was based on the most recent body weight measurement. The first day of dosing was designated as Day 1.

The oral route of exposure was selected because this is a preferred route of human or animal exposure, although other routes of administration may be used.

For the rising-dose phase, the initial dose levels were selected based on information from a 30-day study in dogs wherein FOY-305 (equivalent to Camostat Mesilate) was dosed via oral capsules at dose levels of 10, 30, 100, and 300 mg/kg/day. At 300 mg/kg/day, vomiting was observed which resulted in severely decreased food intake and mortality occurred. Severe gastrointestinal disorders and allotriophagy were observed. There were no significant toxicities observed at the 10, 30, and 100 mg/kg/day dose levels.

Dose levels were modified based on the results of the previously studied dose levels. For the multiple-dose phase, the dose levels were selected based on the results of the rising-dose phase.

### In-Life Procedures, Observations, and Measurements—Rising-Dose Phase

The animals were observed for general health/mortality and moribundity twice daily, once in the morning and afternoon, throughout the study.

The animals were removed from the cage and a detailed clinical observation was performed daily, beginning during Week −1, 1 to 3 hours postdose on days of dosing. Each animal was weighed on the day of randomization and Days 1, 5, 9, 15, 19, and 22. Food consumption was quantitatively measured for each animal daily, starting Week −1, throughout the dosing period, except for on the day of scheduled euthanasia. Electrocardiogram (ECG) measurements were obtained from all animals using leads I, II, III, aV_{R}, aV_{L}, and aV_{F} and a chart speed of 50 mm/second. The ECG measurements were obtained once pretreatment and at approximately 3 hours postdose on Days 1, 5, 9, 15, and 19. Only lead II was evaluated by a board-certified veterinary cardiologist.

Blood was collected by venipuncture of the jugular vein. Urine was collected overnight by cage pan drainage. After collection, samples were transferred to the clinical pathology laboratory for processing. The animals were fasted overnight prior to scheduled clinical pathology sample collections, but had access to water ad libitum. Samples were collected according to Table 5.

TABLE 5
Samples for Clinical Pathology Evaluation

| | | | Clinical | |
|---|---|---|---|---|
| Group Nos. | Time Point | Hematology | Chemistry | Urinalysis |
| All animals | Day −4 | X | X | X |
| 1 | Day 4 | X | X | X |
| 1 | Day 8 | X | X | X |
| 1 | Day 12 | X | X | X |
| 1 | Day 18 | X | X | X |
| 1 | Day 22 | X | X | X |

X = sample collected.

Blood samples were analyzed for the parameters specified in Table 6.

TABLE 6
Hematology Parameters

| | |
|---|---|
| Red blood cell count | White blood cell count |
| Hemoglobin concentration | Neutrophil count (absolute) |
| Hematocrit | Lymphocyte count (absolute) |
| Mean corpuscular volume | Monocyte count (absolute) |
| Red blood cell distribution width | Eosinophil count (absolute) |
| Mean corpuscular hemoglobin | Basophil count (absolute) |
| concentration | Large unstained cells |
| Mean corpuscular hemoglobin | Other cells (as appropriate) |
| Reticulocyte count (absolute) | |
| Platelet count | |

Blood samples were processed for serum, and the serum was analyzed for the parameters specified in Table 7.

TABLE 7
Clinical Chemistry Parameters

| | |
|---|---|
| Alanine aminotransferase | Total protein |
| Aspartate aminotransferase | Albumin |
| Alkaline phosphatase | Globulin (calculated) |
| γ-glutamyltransferase | Albumin/globulin ratio |
| Creatine kinase | Glucose |
| Total bilirubin^{a} | Cholesterol |
| Urea nitrogen | Triglycerides |
| Creatinine | Sodium |
| Calcium | Potassium |
| Phosphorus | Chloride |

Urine samples were processed and analyzed for the parameters listed in Table 8.

TABLE 8
Urinalysis Parameters

| | |
|---|---|
| Color | Protein |
| Appearance/clarity | Glucose |
| Specific gravity | Bilirubin |
| Volume^{a} | Ketones |
| pH | Blood |

Terminal procedures used are summarized in Table 9.

TABLE 9
Terminal Procedures

| | | |
|---|---|---|
| | | Scheduled |
| Group | No. of Animals | Euthanasia |

| No. | Male | Female | Day | Necropsy |
|---|---|---|---|---|
| 1 | 1 | 1 | 22 | X |

X = procedure conducted.

No rising-dose phase animals died during the course of the study.

Rising-dose phase animals surviving until scheduled euthanasia were weighed, samples for evaluation of clinical pathology parameters were collected as specified above, and the animals were euthanized by sodium pentobarbital injection, followed by exsanguination. Animals were fasted overnight before their scheduled necropsy.

Rising-dose phase animals were subjected to a complete necropsy examination, which included evaluation of the carcass and musculoskeletal system; all external surfaces and orifices; cranial cavity and external surfaces of the brain; and thoracic, abdominal, and pelvic cavities with their associated organs and tissues. Necropsy examinations were conducted under the supervision of a board-certified veterinary pathologist.

### In-Life Procedures, Observations, and Measurements—Multiple-Dose Phase

The animals were observed for general health/mortality and moribundity twice daily, once in the morning and afternoon, throughout the study. Cage side observations were performed once daily, beginning during Week −1 and throughout the dosing period. During the dosing period, these observations were performed 1 to 3 hours post the first daily dose. The animals were removed from the cage and a detailed clinical observation was performed weekly, beginning during Week −1. Each animal was weighed on the day of randomization and Days 1, 4, 8, 11, and 15. A fasted weight was recorded on the day of necropsy. Food consumption was quantitatively measured for each animal daily, starting Week −1, throughout the dosing period, except for on the day of scheduled euthanasia.

Electrocardiogram (ECG) measurements were obtained from all animals using leads I, II, III, aV_{R}, aV_{L}, and aV_{F} and a chart speed of 50 mm/second. The ECG measurements were obtained once on Day −1 (pretreatment) and Day 14 (approximately 3 hours post the first daily dose). Only lead II was evaluated by a board-certified veterinary cardiologist.

Blood was collected by venipuncture of the jugular vein. Urine was collected overnight by cage pan drainage. After collection, samples were transferred to the clinical pathology laboratory for processing. The animals were fasted overnight prior to scheduled clinical pathology sample collections, but had access to water ad libitum. Samples were collected according to Table 10.

TABLE 10
Samples for Clinical Pathology Evaluation

| Group | | | Clinical | |
|---|---|---|---|---|
| Nos. | Time Point | Hematology | Chemistry | Urinalysis |
| All animals | Day −39/−11/−4^{a} | X | X | X |
| 1 | Day 15 | X | X | X |

X = sample collected.
^{a}Prior to start of the rising-dose phase, pretest parameters were collected on all animals that were received for study. Because these animals were assigned to the multiple-dose phase, additional pretest clinical pathology data were collected.

Blood samples were analyzed for the parameters specified in Table 11.

TABLE 11
Hematology Parameters

| | |
|---|---|
| Red blood cell count | White blood cell count |
| Hemoglobin concentration | Neutrophil count (absolute) |
| Hematocrit | Lymphocyte count (absolute) |
| Mean corpuscular volume | Monocyte count (absolute) |
| Red blood cell distribution | Eosinophil count (absolute) |
| width | Basophil count (absolute) |
| Mean corpuscular hemoglobin | Large unstained cells |
| concentration | Other cells (as appropriate) |
| Mean corpuscular hemoglobin | |
| Reticulocyte count (absolute) | |
| Platelet count | |

Blood samples were processed for serum, and the serum was analyzed for the parameters specified in Table 12.

TABLE 12
Clinical Chemistry Parameters

| | |
|---|---|
| Alanine aminotransferase | Total protein |
| Aspartate aminotransferase | Albumin |
| Alkaline phosphatase | Globulin (calculated) |
| Gamma-glutamyltransferase | Albumin/globulin ratio |
| Creatine kinase | Glucose |
| Total bilirubin^{a} | Cholesterol |
| Urea nitrogen | Triglycerides |
| Creatinine | Sodium |
| Calcium | Potassium |
| Phosphorus | Chloride |

Urine samples were processed and analyzed for the parameters listed in Table 13.

TABLE 13
Urinalysis Parameters

| | |
|---|---|
| Color | Protein |
| Appearance/clarity | Glucose |
| Specific gravity | Bilirubin |
| Volume^{a} | Ketones |
| pH | Blood |

Terminal procedures are summarized in Table 14.

TABLE 14
Terminal Procedures for Multiple-dose Phase Animals

| | | | Scheduled | Necropsy Procedures | |
|---|---|---|---|---|---|
| Group | No. of Animals | | Euthanasia | | Tissue |
| No. | Male | Female | Day | Necropsy | Collection |
| 1 | 1 | 1 | 15 | X | X |

X = procedure conducted.

No animals died during the course of the study.

Multiple-dose phase animals surviving until scheduled euthanasia were weighed, samples for evaluation of clinical pathology parameters were collected as specified above, and the animals were euthanized by sodium pentobarbital injection, followed by exsanguination. Animals were fasted (overnight) before their scheduled necropsy.

Multiple-dose phase animals were subjected to a complete necropsy examination, which included evaluation of the carcass and musculoskeletal system; all external surfaces and orifices; cranial cavity and external surfaces of the brain; and thoracic, abdominal, and pelvic cavities with their associated organs and tissues. Necropsy examinations were conducted under the supervision of a board-certified veterinary pathologist.

Representative samples of the tissues identified in Table 15 were collected from all multiple-dose phase animals and preserved in 10% neutral buffered formalin, unless otherwise indicated, for possible future evaluation.

TABLE 15
Tissue Collection and Preservation

| | |
|---|---|
| Animal identification | Lung |
| Gland, adrenal | Ovary |
| Gland, pituitary | Pancreas |
| Gland, prostate | Spleen |
| Gland, salivary | Thymus |
| Heart | Urinary bladder |
| | Uterus |

### Computerized Systems

Important computerized systems used in the study are listed.

TABLE 16
Critical Computerized Systems

| | Version | Description of Data Collected |
|---|---|---|
| System Name | No. | and/or Analyzed |
| Provantis | 8 | Applicable in-life and necropsy |
| | | data |
| Systems 600 Apogee | 3.11 | Temperature and/or humidity |
| Insight System | | (animal rooms, refrigerators, |
| | | freezers, and compound storage, |
| | | as applicable) |
| Instem Life Science | 8 | Test material receipt, |
| Systems, DISPENSE | | accountability and/or |
| | | formulation activities |
| Vetronics Computerized | 1.50AT | ECG measurements |
| ECG Analyzer | | |
| Bayer Advia 120 ® | 3.1.8.0 | Hematology data |
| Automated Hematology | | |
| Analyzer | | |
| Olympus AU640e | 7 | Clinical chemistry data |
| Cliniteck Advantus | V1.20 | Urinalysis data |
| Urine Dipstick | | |
| Analyzer | | |

### Results

### Rising Dose Phase

All animals survived to scheduled euthanasia.

At 10 mg/kg, soft feces were noted in the female the day after dosing.

At 30 mg/kg, there were no clinical observations noted.

At 100 mg/kg, clear ocular discharge was observed in the female beginning the day after dosing. In addition, a small amount of the capsule/test article was found in the cage of the female following dosing.

At 200 mg/kg, slight salivation and soft feces/feces with mucoid material were observed in the male and female, along with a continuation of clear ocular discharge in the female.

At 250 mg/kg, severe salivation was observed following dosing in the male and female, along with feces with mucoid material in the male and clear ocular discharge in the female.

There were no notable changes in body weight between doses.

There were no notable changes in food consumption between doses.

All dogs maintained sinus rhythms throughout the study. A left axis deviation shift was noted in the rising-dose male dog after dose cycle 1 (10 mg/kg). This axis change is occasionally seen in normal dogs and was not test article related. No ventricular premature depolarizations were noted throughout the study. Heart rate, PR interval, QRS duration, QT interval, and QTc were all within normal limits during the ECGs recorded.

There were no toxicologically meaningful changes in hematology parameters following each dose.

### Clinical Chemistry

Potential test article-related changes in clinical chemistry parameters were limited to increased creatinine, total bilirubin, and urea nitrogen in the male following the 250 mg/kg dose and increased creatine kinase in the female following the 200 and 250 mg/kg doses.

There were no toxicologically meaningful changes in urinalysis parameters following each dose.

There were no gross necropsy findings on Day 22.

### Multiple-Dose Phase

All animals survived to scheduled euthanasia on Day 15.

### Clinical Observations

In the male, clinical observations were limited to thin appearance and a single occurrence of vomitus. In the female, clinical observations were limited to clear ocular discharge and single occurrences of vomitus and mucoid feces.

Body weight loss was observed in both the male (−9.2%) and female (−2.7%) from Days 1 to 15.

There were no notable changes in food consumption during the study.

All dogs maintained sinus rhythms throughout the study. An atrial premature depolarization was noted in the male dog prior to treatment. This rhythm disturbance is occasionally seen in normal dogs. A left axis deviation shift was noted in the male dog on the last day of dosing (Day 14). This axis change is occasionally seen in normal dogs and was not test article related. No ventricular premature depolarizations were noted throughout the study. Heart rate, PR interval, QRS duration, QT interval, and QTc were all within normal limits during the ECGs recorded.

There were no toxicologically meaningful changes in hematology parameters on Day 15 compared to pretest values.

### Clinical Chemistry

Potential test article-related changes in clinical chemistry values were limited to increased creatinine and γ-glutamyl transferase in the male and female on Day 15.

There were no toxicologically meaningful changes in urinalysis parameters on Day 15 compared to pretest values.

There were no gross necropsy findings on Day 15.

### Conclusion

In conclusion, oral administration of Camostat Mesilate 3 times per day at 200 mg/kg/day for 14 days resulted in body weight loss and potential test article-related increases in creatinine and γ-glutamyl transferase. Based on these results, 200 mg/kg/day was chosen as the high-dose level for this test article.

### Example 3

### A Rising-Dose and Multiple-Dose Tolerance Study of Camostat Mesilate by Oral Gavage Administration in Rats

The objective of this study was to determine the potential toxicity of Camostat Mesilate, a potential therapeutic agent for pancreatitis, when given orally as a single dose to rats followed by at least a 7-day observation period repeated for up to 4 cycles. In addition, the potential toxicity of Camostat Mesilate was determined when given orally 3 times a day for 7 days to rats.

The study design was as follows:

TABLE 17
Experimental Design for the Rising-dose Study

| | | | | Dose | No. of Animals | |
|---|---|---|---|---|---|---|
| | | Dose | Dose | Concen- | Rising-dose | |
| Group | Test | Level | Volume | tration | Study | |
| No. | Material | (mg/kg) | (mL/kg) | (mg/mL) | Males | Females |
| 1 | Camostat | 100 | 10 | 10 | — | 3 |
| | Mesilate | | | | | |
| 2 | Camostat | 550 | 10 | 55 | — | 3 |
| | Mesilate | | | | | |
| 3 | Camostat | 1300 | 10 | 130 | — | 3 |
| | Mesilate | | | | | |
| 4 | Camostat | 1300 | 10 | 130 | 3 | — |
| | Mesilate | | | | | |

The following parameters and end points were evaluated in the rising-dose phase of this study: clinical signs, body weights, body weight changes, food consumption, and gross necropsy findings.

TABLE 18
Experimental Design for the Multiple-dose Study

| | | Dose | Dose | Dose | Dose | No. of Animals | |
|---|---|---|---|---|---|---|---|
| | | Level | Level | Volume | Concen- | Multiple-dose | |
| Group | Test | (mg/kg/ | (mg/kg/ | (mL/kg/ | tration | Study | |
| No. | Material | day) | dose) | dose) | (mg/mL) | Males | Females |
| 1 | RODI | 0 | 0 | 5 | 0 | 3 | 3 |
| | Water | | | | | | |
| 2 | Camostat | 100 | 33.5 | 5 | 6.7 | 3 | 3 |
| | Mesilate | | | | | | |
| 3 | Camostat | 550 | 183.5 | 5 | 36.7 | 3 | 3 |
| | Mesilate | | | | | | |
| 4 | Camostat | 1300 | 433.5 | 5 | 86.7 | 3 | 3 |
| | Mesilate | | | | | | |

The following parameters and end points were evaluated in the multiple-dose phase of this study: clinical signs, body weights, body weight changes, food consumption, and gross necropsy findings.

Rising-dose Phase: All animals survived to scheduled euthanasia on Day 8 and there were no test article-related clinical observations. All animals gained weight, there were no significant changes in food consumption, and there were no significant gross necropsy findings at 100, 550, or 1300 mg/kg. Based on these results, the same dose levels were tested in the multiple-dose phase.

Multiple-dose Phase: All animals survived to scheduled euthanasia on Day 8. Most of the 1300 mg/kg/day animals were noted to be thin by the end of the study. Food consumption was lower in the 1300 mg/kg/day males (−31%) and females (−34%) as compared to controls, correlating with body weight loss in the males (−15%) and females (−13%) from Days 1 to 8. There were no toxicologically meaningful gross necropsy findings.

In conclusion oral administration of Camostat Mesilate 3 times per day for 7 days resulted in body weight loss and decreased food consumption. Because there were no adverse clinical signs or gross necropsy findings associated with the body weight loss, 1300 mg/kg/day was chosen as a suitable high-dose level with this test article.

### Materials and Methods

### Test Article

Identification: Camostat Mesilate
Physical Description: White powder
Purity: 100% (assumed for calculation purposes)
Storage Conditions: Kept in a refrigerator set to maintain 5° C.
Supplier: Stason Pharmaceuticals

### Control Article

Identification: Reverse Osmosis Deionized (RODI) Water
Supplier: Obtained from the Testing Facility tap

For each batch (lot) of test article, a reserve sample (1 g) was collected and maintained under the appropriate storage conditions.

The control article, RODI Water, was administered as obtained from the Testing Facility tap. The control article was also stirred continuously during dosing.

Test article dosing formulations were prepared at appropriate concentrations to meet dose level requirements. The dosing formulations were prepared prior to each dose administration for the rising-dose study. For the multiple-dose study, dosing formulations were prepared once, stored in a refrigerator set to maintain 5° C., and dispensed on the days of dosing. The dosing formulations were removed from the refrigerator and stirred for at least 30 minutes before dosing. The dosing formulations were also stirred continuously during dosing.

Dose formulation samples were collected for analysis as indicated in Table 19.

TABLE 19
Dose Formulation Sample Collection Schedule

| Period | Interval | Concentration | Homogeneity | Stability |
|---|---|---|---|---|
| Multiple-dose | Day −1 | All groups^{a} | Groups 2 | N/A |
| | | | and 4^{a} | |

N/A = not applicable.
^{a}The homogeneity results obtained from the top, middle, and bottom for the Group 2 and 4 preparations were averaged and utilized as the concentration results.

Samples were transferred under ambient conditions to the analytical laboratory at the Testing Facility for analysis. Analyses described below were performed by HPLC using a validated analytical procedure.

Duplicate sets of samples from the top, middle, and bottom (1 mL each) for each sampling time point were sent to the analytical laboratory. On days where only concentration analysis was required, the formulation was only sampled from the middle. Concentration results were considered acceptable if mean sample concentration results were within or equal to 10% of theoretical concentration. Each individual sample concentration result was considered acceptable if it was within or equal to 15%. Homogeneity results were considered acceptable if the relative standard deviation of the mean value at each sampling location was ≦5%. After acceptance of the analytical results, backup samples were discarded.

Stability analyses were performed to demonstrate that the test article is stable in the vehicle when prepared and stored under the same conditions at concentrations bracketing those used in the present study

A total of 18 male and 24 female Han Wistar rats were received from Charles River Laboratories, Raleigh, N.C. The animals were examined and weighed on the day following receipt. The rising-dose phase animals were 8 weeks old and weighed between 176 g and 255 g at initiation of dosing and the multiple-dose phase animals were 8 weeks old and weighed between 172 g and 259 g at initiation of dosing.

The Han Wistar rat was chosen as the animal model for this study because it is an accepted rodent species for preclinical toxicity testing by regulatory agencies.

The total number of animals used in this study was considered to be the minimum required to properly characterize the effects of the test article. This study has been designed such that it does not require an unnecessary number of animals to accomplish its objectives.

Studies in laboratory animals provide the best available basis for extrapolation to humans and are required to support regulatory submissions. Acceptable models which do not use live animals currently do not exist.

Each animal was identified by a cage card and ear tag after randomization.

The animals were acclimated to their designated housing for at least 6 days for the rising-dose phase and 10 days for the multiple-dose phase before the first day of dosing.

Animals assigned to study were assigned to groups by a stratified randomization scheme designed to achieve similar group mean body weights. Males and females were randomized separately. Animals in poor health or at extremes of body weight range were not assigned to groups.

On arrival, animals were individually housed until randomization. Following randomization, animals were group housed (up to 3 animals of the same sex and same dosing group together) in polycarbonate cages containing appropriate bedding equipped with an automatic watering valve. Housing and care were as specified in the USDA Animal Welfare Act (9 CFR, Parts 1, 2, and 3) and as described in the Guide for the Care and Use of Laboratory Animals from the National Research Council.¹ ¹. Guide for the care and use of laboratory animals. Washington, D.C.: National Academy Press. NRC (National Research Council); 2011.

Temperatures of 69° F. to 71° F. (21° C. to 22° C.) with a relative humidity of 45% to 56% were maintained. A 12-hour light/12-hour dark cycle was maintained, except when interrupted for designated procedures. Ten or greater air changes per hour with 100% fresh air (no air recirculation) were maintained in the animal rooms.

PMI Nutrition International Certified Rodent Chow No. 5CR4 (14% protein) was provided ad libitum throughout the study, except during designated procedures. The lot number and expiration date of each batch of diet used during the study were recorded. The feed was analyzed by the supplier for nutritional components and environmental contaminants. Results of the dietary analyses were provided by the manufacturer for each lot of diet and are on file at the Testing Facility. Based on the results of these analyses, there were no contaminants that would interfere with the conduct or interpretation of the study.

Municipal tap water, after treatment by reverse osmosis and ultraviolet irradiation, was freely available to each animal via an automatic watering system (except during designated procedures). Periodic analysis of the water is performed, and results of these analyses are on file at the Testing Facility. It is considered that there are no known contaminants in the water that could interfere with the outcome of the study.

For psychological/environmental enrichment, animals were provided with items such as a chewing object, except when interrupted by study procedures/activities.

Veterinary care was available throughout the course of the study; however, no treatments were required.

### Experimental Design

TABLE 20
Experimental Design for the Rising-dose Study

| | | | | Dose | No. of Animals | |
|---|---|---|---|---|---|---|
| | | Dose | Dose | Concen- | Rising-dose | |
| Group | Test | Level | Volume | tration | Study | |
| No. | Material | (mg/kg) | (mL/kg) | (mg/mL) | Males | Females |
| 1 | Camostat | 100 | 10 | 10 | — | 3 |
| | Mesilate | | | | | |
| 2 | Camostat | 550 | 10 | 55 | — | 3 |
| | Mesilate | | | | | |
| 3 | Camostat | 1300 | 10 | 130 | — | 3 |
| | Mesilate | | | | | |
| 4 | Camostat | 1300 | 10 | 130 | 3 | — |
| | Mesilate | | | | | |

TABLE 21
Experimental Design for the Multiple-dose Study

| | | Dose | Dose | Dose | Dose | No. of Animals | |
|---|---|---|---|---|---|---|---|
| | | Level | Level | Volume | Concen- | Multiple-dose | |
| Group | Test | (mg/kg/ | (mg/kg/ | (mL/kg/ | tration | Study | |
| No. | Material | day) | dose) | dose) | (mg/mL) | Males | Females |
| 1 | RODI | 0 | 0 | 5 | 0 | 3 | 3 |
| | Water | | | | | | |
| 2 | Camostat | 100 | 33.5 | 5 | 6.7 | 3 | 3 |
| | Mesilate | | | | | | |
| 3 | Camostat | 550 | 183.5 | 5 | 36.7 | 3 | 3 |
| | Mesilate | | | | | | |
| 4 | Camostat | 1300 | 433.5 | 5 | 86.7 | 3 | 3 |
| | Mesilate | | | | | | |

For the rising-dose study, the test article was administered to the appropriate animals as a single dose by oral gavage. At least a 3-day observation period was maintained before proceeding to the next dose level/group. The dose volume for each animal was based on the most recent body weight measurement. The doses were given using a syringe with attached gavage cannula. The first day of dosing for each group was designated as Study Day 1.

For the multiple-dose study, the test and control articles were administered to the appropriate animals 3 times a day by oral gavage from Days 1 to 7. The doses were administered 8 hours±30 minutes apart. The dose volume for each animal was based on the most recent body weight measurement. The doses were given using a syringe with attached gavage cannula. The first day of dosing was designated as Study Day 1.

The dosing formulations were stirred continuously during dose administration.

The oral route of exposure was selected because this is the intended route of human exposure.

For the rising-dose study, the initial dose levels were selected based on information provided by the Sponsor. In a previous study, FOY-305 (equivalent to Camostat Mesilate) was dosed orally to rats for 90 and 180 days at doses of 100, 235, 550, and 1300 mg/kg/day. In all test article-treated groups, hypersalivation was observed immediately after dosing. At 1300 mg/kg/day, preening and a slight decrease in locomotor activity were also observed up to 30 minutes postdose. As the number of doses increased, the 550 and 1300 mg/kg/day groups showed aversive behavior for dosing. Body weight gain was suppressed at 550 and 1300 mg/kg/day. Pancreas weights were increased in all test article-treated groups. At the end of the recovery period, pancreas weights were still increased; however, not to the extent as observed at the end of 90 and 180 days of dosing. In all test article-treated groups, pancreatitis with acinar cell hypertrophy associated with increased zymogen granule content in the cells was observed histopathologically. In addition, acinar cell vacuolation was observed at 550 and 1300 mg/kg/day. Following the recovery period, there were no significant differences in histopathology between the control and test article-treated groups.

For the multiple-dose study, the dose levels were selected based on the results of the rising-dose study.

The multiple-dose study was dosed 3 times a day to mimic the dosing regimen planned for the clinic.

### In-Life Procedures, Observations, and Measurements—Rising-Dose Study

The animals were observed for general health/mortality and moribundity twice daily, once in the morning and afternoon, throughout the study. The animals were removed from the cage and a detailed clinical observation was performed on the day of randomization and Days 1 to 8; 1 to 3 hours postdose on the day of dosing. Each animal was weighed on the day of randomization and Days 1 to 8. Food consumption was quantitatively measured for each animal weekly, starting Day −3, throughout the study. Terminal procedures are summarized in Table 22.

TABLE 22
Terminal Procedures - Rising-dose Study

| | | |
|---|---|---|
| | | Scheduled |
| Group | No. of Animals | Euthanasia |

| No. | Male | Female | Day | Necropsy |
|---|---|---|---|---|
| 1 | — | 3 | 8 | X |
| 2 | — | 3 | | |
| 3 | — | 3 | | |
| 4 | 3 | — | | |

X = procedure conducted.

No animals died during the course of the study. Rising-dose study animals surviving until scheduled euthanasia had a terminal body weight recorded and the animals were euthanized by carbon dioxide inhalation, followed by exsanguination.

Rising-dose study animals were subjected to a complete necropsy examination, which included evaluation of the carcass and musculoskeletal system; all external surfaces and orifices; cranial cavity and external surfaces of the brain; and thoracic, abdominal, and pelvic cavities with their associated organs and tissues. Necropsy examinations were conducted under the supervision of a board-certified veterinary pathologist.

### In-Life Procedures, Observations, and Measurements—Multiple-Dose Study

The animals were observed for general health/mortality and moribundity twice daily, once in the morning and afternoon, throughout the study.

### Clinical Observations

Cage side observations were performed once daily, beginning during Week −1 and throughout the dosing phase. During the dosing period, these observations were performed 1 to 3 hours post the first daily dose.

The animals were removed from the cage and a detailed clinical observation was performed on the day of randomization and Days 1 and 8. Each animal was weighed on the day of randomization (Day −4) and Days 1 to 8. Food consumption was quantitatively measured for each animal weekly, starting Day −4, throughout the dosing phase. Terminal procedures are summarized in Table 23.

TABLE 23
Terminal Procedures - Multiple-dose Study

| | | | Scheduled | Necropsy Procedures | |
|---|---|---|---|---|---|
| Group | No. of Animals | | Euthanasia | | Tissue |
| No. | Male | Female | Day | Necropsy | Collection |
| 1 | 3 | 3 | 8 | X | X |
| 2 | 3 | 3 | | | |
| 3 | 3 | 3 | | | |
| 4 | 3 | 3 | | | |

X = procedure conducted.

No animals died during the course of the study. Multiple-dose study animals surviving until scheduled euthanasia had a terminal body weight recorded and the animals were euthanized by carbon dioxide inhalation, followed by exsanguination. When possible, the animals were euthanized rotating across dose groups such that similar numbers of animals from each group, including controls, were necropsied throughout the day.

Multiple-dose study animals were subjected to a complete necropsy examination, which included evaluation of the carcass and musculoskeletal system; all external surfaces and orifices; cranial cavity and external surfaces of the brain; and thoracic, abdominal, and pelvic cavities with their associated organs and tissues. Necropsy examinations were conducted under the supervision of a board-certified veterinary pathologist.

Representative samples of the tissues identified in Table were collected from all animals and preserved in 10% neutral buffered formalin for possible future evaluation.

TABLE 24
Tissue Collection and Preservation

| | |
|---|---|
| Animal identification | Lung |
| Gland, adrenal | Ovary |
| Gland, pituitary | Pancreas |
| Gland, prostate | Spleen |
| Gland, salivary | Thymus |
| Gland, seminal vesicle | Urinary bladder |
| Heart | Uterus |

### Computerized Systems

Computerized systems used in the study are listed below. All computerized systems used in the conduct of this study have been validated; when a particular system has not satisfied all requirements, appropriate administrative and procedural controls were implemented to assure the quality and integrity of data.

TABLE 25
Critical Computerized Systems

| | Version | Description of Data Collected |
|---|---|---|
| System Name | No. | and/or Analyzed |
| Provantis | 8 | Applicable in-life and necropsy |
| | | data |
| Systems 600 Apogee | 3.11 | Temperature and/or humidity |
| Insight System | | (animal rooms, refrigerators, |
| | | freezers, and compound storage, |
| | | as applicable) |
| Instem Life Science | 8 | Test material receipt, |
| Systems, DISPENSE | | accountability and/or |
| | | formulation activities |
| Empower 3 | Build 3471, | Analytical analysis |
| | SR1 | |
| In-house reporting | NEVIS 2 | In-life (multiple-dose phase |
| software Nevis | (SAS 9.2) | body weights, food |
| (using SAS) | | consumption) |

### Statistical Analysis

For the rising-dose study, data were presented as individual values by animal. Descriptive statistics number, mean, and standard deviation were reported.

For the multiple-dose study, numerical data collected on scheduled occasions for the listed variables were analyzed as indicated according to sex and occasion. Descriptive statistics number, mean and standard deviation (or % CV or SE when deemed appropriate) were reported whenever possible. Inferential statistics were performed according to the matrix below when possible, but excluded semi-quantitative data, and any group with less than 3 observations.

TABLE 26
Statistical Matrix

| Variables for | Statistical Method |
|---|---|
| Inferential Analysis | ANOVA |
| Body Weight | X |
| Food Consumption | X |
| Body Weight Change | X |

The following pairwise comparisons were made:

Group 2 vs. Group 1
Group 3 vs. Group 1
Group 4 vs. Group 1

Levene's test was used to assess the homogeneity of group variances parametric assumption at the 5% significance level. Datasets with at least 3 groups were compared using an overall one-way ANOVA F-test or Kruskal-Wallis test (if parametric assumptions were not met) at the 5% significance level. The above pairwise comparisons were conducted using a two-sided Dunnett's or Dunn's test, respectively, if the overall test was significant. Datasets with 2 groups were compared using a two-sided t-test or Wilcoxon Rank-Sum test, respectively. All significant pairwise comparisons were reported at the 0.1%, 1%, and 5% significance levels.

### Results

All study samples analyzed had mean concentrations within or equal to the acceptance criteria of ±10% (individual values within or equal to ±15%) of their theoretical concentrations. For homogeneity, the RSD of concentrations for all samples in each group tested was within the acceptance criterion of ≦5%.

### Rising-Dose Phase

All animals survived to scheduled euthanasia on Day 8.

### Clinical Observations

There were no test article-related clinical observations noted. At 550 mg/kg, hair loss was observed in 1 animal; however, this was not considered test article related because hair loss is a common finding in laboratory rats. At 1300 mg/kg, piloerection and abnormal breathing sounds were observed in 1 animal on Day 2. An undetermined amount of test article was expelled during dosing in this animal on Day 1; therefore, these clinical signs could be a result of aspiration of the test article during dosing, and were not considered to be a direct test article effect. All animals gained weight from Days 1 to 8 and there were no significant changes in food consumption during the study.

There were no significant gross necropsy findings observed. Dark discoloration of the thymus was observed in one 1300 mg/kg male; however, this was not considered test article related because it is a common finding in laboratory rats.

### Multiple-Dose Phase

All animals survived to scheduled euthanasia on Day 8 and there were no clinical observations noted in the 0, 100, or 550 mg/kg/day animals. In the 1300 mg/kg/day group, 3/3 males and 2/3 females were noted to be thin on Day 8.

At 1300 mg/kg/day, body weight loss was noted from Days 1 to 8 (−15% in the males and −13% in the females). In addition, mean body weights were statistically decreased as compared to controls from Days 4 to 8 in the males and from Days 5 to 8 in the females, with body weights that were 25% and 19% lower than controls in males and females on Day 8, respectively.

There were no statistically significant or toxicologically meaningful differences in mean body weights in the 100 or 550 mg/kg/day groups as compared to controls, and mean body weights were within 4.4% of controls on Day 8.

There were no statistically significant differences in mean food consumption from Days 1 to 7; however, food consumption was 31% and 34% lower than controls in the 1300 mg/kg/day males and females, respectively, correlating with the decreased body weights.

There were no toxicologically meaningful gross necropsy findings noted during the study.

A small seminal vesicle was noted in one 1300 mg/kg/day male. Due to the low incidence, the lack of histopathological examination, and the likelihood that this was related to the sexual maturity of the animal, this was not considered test article related.

### Conclusion

In conclusion, oral administration of Camostat Mesilate 3 times per day for 7 days resulted in body weight loss and decreased food consumption. Because there were no adverse clinical signs or gross necropsy findings associated with the body weight loss, 1300 mg/kg/day was chosen to be the high-dose level for this test article.

1. A method for treating canine pancreatitis comprising: measuring the serum concentration of canine pancreas-specific lipase and administering a dose of Camostat Mesilate of from about 8 to about 200 mg/kg in 24 hrs when the dog has a serum concentration of about 400 μg/L or more of canine pancreas-specific lipase.

2. The method for treating canine pancreatitis of claim 1, wherein the dosage is from about 24 mg/kg to about 200 mg/kg in 24 hrs.

3. The method for treating canine pancreatitis of claim 1, further comprising administering a dose of Camostat Mesilate about every 8-12 hrs.

4. The method for treating canine pancreatitis of claim 1, further comprising administering a dose of from about 8 mg/kg to about 66.7 mg/kg about every 8 hrs.

5. The method for treating canine pancreatitis of claim 1, wherein the dosage is administered orally.

6. The method for treating canine pancreatitis of claim 1, wherein the dog is treated for a period of between 7 to 180 days.

7. The method for treating canine pancreatitis of claim 1, wherein the dog is treated for as long as its quality of life is improved.

8. The method for treating canine pancreatitis of claim 1, wherein the Camostat Mesilate is administered in a capsule or tablet dosage form.

9. The method for treating canine pancreatitis of claim 1, wherein the Camostat Mesilate is administered at a dosage of 20, 40, 60, 80±10 mgs; 100, 150, 200, 250, 300, 350, 400, 450, 500, 550, 600, 650±25 mgs; 700, 800, 900, 1000, 1100±50 mgs, 1200, 1400, 1600, or 1800±100 mgs.

10. The method for treating canine pancreatitis of claim 1, wherein the Camostat Mesilate is administered in banded weight ranges as follows: for a target dose of 50 mg/kg/day a dose of 300 mg twice a day for dogs weighing up to about 18 kg, a dose of 600 mg twice a day for dogs weighing between about 19 to about 33 kgs, a dose of 900 mg twice a day for dogs weighing between about 34 to about 45 kgs, and a dose of 1200 mgs twice a day for dogs weighing over 46 kgs.

11. A method for treating pancreatitis comprising identifying a patient with pancreatitis and administering to the patient a daily dose of Camostat Mesilate of from about 8 to about 1300 mg/kg or administering an equivalent amount of 4-(4-guanidino-benzoyloxy) phenylacetic acid.

12. The method for treating pancreatitis wherein the dose of Camostat Mesilate or 4-(4-guanidino-benzoyloxy) phenylacetic acid is administered in a single dose.

13. The method for treating pancreatitis of claim 11 wherein the daily dose of Camostat Mesilate or 4-(4-guanidino-benzoyloxy) phenylacetic acid is given in multiple administrations.

14. The method for treating pancreatitis of claim 11 wherein the daily dose of Camostat Mesilate or 4-(4-guanidino-benzoyloxy) phenylacetic acid is given for about 7 to about 180 days.

15. The method for treating pancreatitis of claim 11 wherein the daily dose of Camostat Mesilate is from about 24 to about 200 mg/kg or an equivalent amount of 4-(4-guanidino-benzoyloxy) phenylacetic acid for a dog.

16. The method for treating pancreatitis of claim 11 wherein the daily dose of Camostat Mesilate is from about 24 to about 200 mg/kg for a dog and is administered for about 7 to 180 days.

17. The method for treating pancreatitis of claim 11 wherein the daily dose of Camostat Mesilate is from about 24 to about 200 mg/kg for a dog after administration three times daily and wherein the administration is repeated for at least 7 days.

18. The method for treating pancreatitis of claim 11 wherein the Camostat Mesilate or 4-(4-guanidino-benzoyloxy) phenylacetic acid is administered in an oral capsule or tablet.

19. The method for treating pancreatitis of claim 11 wherein the Camostat Mesilate or 4-(4-guanidino-benzoyloxy) phenylacetic acid is administered orally.

20. A method for reducing the serum cPLI below 400 μg/L in a dog having a serum cPLI of at least 400 μg/L comprising: measuring the serum concentration of canine pancreas-specific lipase in a dog and administering a dose of Camostat Mesilate of from about 8 to about 200 mg/kg in 24 hrs when the dog has a serum concentration of 400 μg/L or more of cPLI.
